(19) United States
(12) Patent Application Publication
(10) Pub. No.: US 2018/0146847 A1
(43) Pub. Date: May 31, 2018

(54) IMAGE PROCESSING DEVICE, IMAGING SYSTEM, IMAGE PROCESSING METHOD, AND COMPUTER-READABLE RECORDING MEDIUM

(71) Applicant: OLYMPUS CORPORATION, Tokyo (JP)

(72) Inventor: Takeshi OTSUKA, Tokyo (JP)

(73) Assignee: OLYMPUS CORPORATION, Tokyo (JP)

(21) Appl. No.: 15/865,372

(22) Filed: Jan. 9, 2018

Related U.S. Application Data
(63) Continuation of application No. PCT/JP2015/070459, filed on Jul. 16, 2015.

(51) Int. Cl. A61B 1/05; G06T 7/00; G06T 7/50

(52) U.S. Cl. CPC A61B 1/05; G06T 2207/30101; G06T 7/50; G06T 7/0012

(57) ABSTRACT

An image processing device includes: an absorbance calculating unit configured to calculate absorbances at wavelengths based on pixel values of a plurality of pixels constituting an image; a component amount estimating unit configured to estimate amounts of two or more kinds of light absorbing components contained respectively in two or more kinds of tissue including specified tissue by using an absorbance in a first wavelength band among the absorbances at the wavelengths calculated by the absorbance calculating unit, the first wavelength band being a part of the wavelengths; an estimation error calculating unit configured to calculate estimation errors caused by the component amount estimating unit; and a depth estimating unit configured to estimate a depth at which the specified tissue is present in the object based on an estimation error in a second wavelength band with shorter wavelength than the first wavelength band among the estimation errors.

### CROSS-REFERENCE TO RELATED APPLICATION

This application is a continuation of International Application No. PCT/JP2015/070459, filed on Jul. 16, 2015, the entire contents of which are incorporated herein by reference.

### BACKGROUND

The present disclosure relates to an image processing device, an imaging system, an image processing method, and a computer-readable recording medium.

One example of physical quantities representing a physical property inherent to an object is a spectral transmittance spectrum. Spectral transmittance is a physical quantity representing a ratio of transmitted light to incident light at each wavelength. While RGB values in an image obtained by capturing an object are information depending on a change in illumination light, camera sensitivity characteristics, and the like, spectral transmittance is information inherent to an object whose value is not changed by exogenous influences. Spectral transmittance is therefore used as information for reproducing original colors of an object in various fields.

Multiband imaging is known as means for obtaining a spectral transmittance spectrum. In multiband imaging, an object is captured by the frame sequential method while 16 bandpass filters, through which illumination light is transmitted, are switched by rotation of a filter wheel, for example. As a result, a multiband image with pixel values of 16 bands at each pixel position is obtained.

Examples of a technique for estimating a spectral transmittance from such a multiband image include an estimation technique using the principal component analysis and an estimation technique using the Wiener estimation. The Wiener estimation is a technique known as one of linear filtering techniques for estimating an original signal from an observed signal with superimposed noise, and minimizes error in the light of the statistical properties of the observed object and the characteristics of noise in observation. Since some noise is contained in a signal from a camera capturing an object, the Wiener estimation is highly useful as a technique for estimating an original signal.

With respect to a point x at a given pixel position in a multiband image, a pixel value g(x,b) in a band b and a spectral transmittance t(x,λ) of light having a wavelength λ at a point on an object corresponding to the point x satisfy the relation of the following formula (1) based on a camera response system.

g(x,b)=∫_{λ}f(b,λ)·s(λ)·e(λ)·t(x,λ)dλ+nₛ(b) (1)

In the formula (1), a function f(b,λ) represents a spectral transmittance of light having the wavelength λ at a b-th bandpass filter, a function s(λ) represents a spectral sensitivity characteristic of a camera at the wavelength λ, a function e(λ) represents a spectral radiation characteristic of illumination at the wavelength λ, and a function nₛ(b) represents observation noise in the band b. Note that a variable b for identifying a bandpass filter is an integer satisfying 1≤b≤16 in the case of 16 bands, for example.

In actual calculation, a relational formula (2) of a matrix obtained by discretizing the wavelength λ is used instead of the formula (1).

G(x)=FSET(x)+N (2)

When the number of sample points in the wavelength direction is represented by m and the number of bands is represented by n, a matrix G(x) is a matrix with n rows and one column having pixel values g(x,b) at points x as elements, a matrix T(x) is a matrix with m rows and one column having spectral transmittances t(x,λ) as elements, and a matrix F is a matrix with n rows and m columns having spectral transmittances f(b,λ) of the filters as elements in the formula (2). In addition, a matrix S is a diagonal matrix with m rows and m columns having spectral sensitivity characteristics s(λ) of the camera as diagonal elements. A matrix E is a diagonal matrix with m rows and m columns having spectral radiation characteristics e(λ) of the illumination as diagonal elements. A matrix N is a matrix with n rows and one column having observation noise nₛ(b) as elements. Note that, since the formula (2) summarizes formulas on a plurality of bands by using matrices, the variable b identifying a bandpass filter is not described. In addition, integration concerning the wavelength λ is replaced by a product of matrices.

Here, for simplicity of description, a matrix H defined by the following formula (3) is introduced. The matrix H is also called a system matrix.

H=FSE (3)

As a result of using the system matrix H, the formula (2) is replaced by the following formula (4).

G(x)=HT(x)+N (4)

For estimation of the spectral transmittance at each point of the object based on a multiband image by using the Wiener estimation, spectral transmittance data T̂(x), which are estimated values of the spectral transmittance, are given by a relational formula (5) of matrices. In the formula, a symbol T̂ means that a symbol “̂(hat)” representing an estimated value is present over a symbol T. The same applies below.

\hat{T}(x)=WG(x) (5)

A matrix W is called a “Wiener estimation matrix” or an “estimation operator used in the Wiener estimation,” and given by the following formula (6).

W=R_{SS}H^{T}(HR_{SS}H^{T}+R_{NN})⁻¹ (6)

In the formula (6), a matrix R_{SS} is a matrix with m rows and m columns representing an autocorrelation matrix of the spectral transmittance of the object. A matrix R_{NN} is a matrix with n rows and n columns representing an autocorrelation matrix of noise of the camera used for imaging. With respect to a given matrix X, a matrix X^{T} represents a transposed matrix of the matrix X and matrix X⁻¹ represents an inverse matrix of the matrix X. The matrices F, S, and E (see the formula (3)) constituting the system matrix H, that is, the spectral transmittances of the filters, the spectral sensitivity characteristics of the camera, and the spectral radiation characteristics, the matrix R_{SS}, and the matrix R_{NN} are obtained in advance.

Note that, for observation of a thin, translucent object with transmitted light, it is known that dye amounts of an object may be estimated based on the Lambert-Beer law since absorption is dominant in optical phenomena. Hereinafter, a method of observing a stained sliced specimen as an object with a transmission microscope and estimating a dye amount at each point of the object will be explained. More particularly, the dye amounts at points on the object corresponding to respective pixels are estimated based on the spectral transmittance data T̂(x). Specifically, a hematoxylin and eosin (HE) stained object is observed, and estimation is performed on three kinds of dyes, which are hematoxylin, eosin staining cytoplasm, and eosin staining red blood cells or an intrinsic pigment of unstained red blood cells. These names of dyes will hereinafter be abbreviated as a dye H, a dye E, and a dye R. Technically, red blood cells have their own color in an unstained state, and the color of the red blood cells and the color of eosin that has changed during the HE staining process are observed in a superimposed state after the HE staining. Thus, to be exact, combination of the both is referred to as the dye R.

Typically, when the object is a light transmissive material, the intensity I₀(λ) of incident light and the intensity I(λ) of outgoing light at each wavelength λ are known to satisfy the Lambert-Beer law expressed by the following formula (7).

$$\frac{I ( λ )}{{I}_{0} ( λ )} = {e}^{- k ( λ ) \cdot {d}_{0}} \qquad ( 7 )$$

In the formula (7), a symbol k(λ) represents a coefficient unique to a material determined depending on the wavelength λ, and a symbol d₀ represents the thickness of the object.

The left side of the formula (7) means the spectral transmittance t(λ), and the formula (7) is replaced by the following formula (8).

t(λ)=e^{-k(λ)d}^{₀} (8)

In addition, spectral absorbance a(λ) is given by the following formula (9).

a(λ)=k(λ)·d₀ (9)

With the formula (9), the formula (8) is replaced by the following formula (10).

t(λ)=e^{-a(λ)} (10)

When the HE stained object is stained by three kinds of dyes, which are the dye H, the dye E, and the dye R, the following formula (11) is satisfied at each wavelength λ based on the Lambert-Beer law.

$$\frac{I ( λ )}{{I}_{0} ( λ )} = {e}^{- ( {k}_{H} ( λ ) \cdot {d}_{H} + {k}_{E} ( λ ) \cdot {d}_{E} + {k}_{R} ( λ ) \cdot {d}_{R} )} \qquad ( 11 )$$

In the formula (11) coefficients k_{H}(λ), k_{E}(λ), and k_{R}(λ) are coefficients respectively associated with the dye H, the dye E, and the dye R. These coefficients k_{H}(λ), k_{E}(λ), and k_{R}(λ) correspond to dye spectra of the respective dyes staining the object. These dye spectra will hereinafter be referred to as reference dye spectra. Each of the reference dye spectra k_{H}(λ), k_{E}(λ), and k_{R}(λ) may be easily obtained by application of the Lambert-Beer law, by preparing in advance specimens individually stained with the dye H, the dye E, and the dye R and measuring the spectral transmittance of each specimen by a spectroscope.

In addition, symbols d_{H}, d_{E}, and d_{R} are values representing virtual thicknesses of the dye H, the dye E, and the dye R at points of the object respectively corresponding to pixels constituting a multiband image. Since dyes are normally found scattered across an object, the concept of thickness is not correct; however, “thickness” may be used as an index of a relative dye amount indicating how much a dye is present as compared to a case where the object is assumed to be stained with a single dye. In other words, the values d_{H}, d_{E}, and d_{R} may be deemed to represent the dye amounts of the dye H, the dye E, and the dye R, respectively.

When the spectral transmittance at a point of the object corresponding to a point x on the image is represented by t(x,λ), the spectral absorbance is represented by a(x,λ), and the object is stained with three dyes, which are the dye H, the dye E, and the dye R, the formula (9) is replaced by the following formula (12).

a(x,λ)=k_{H}(λ)·d_{H}+k_{E}(λ)·d_{E}+k_{R}(λ)·d_{R} (12)

When the estimated spectral transmittance and the estimated absorbance at the wavelength λ of the spectral transmittance T̂(x) are represented by t̂(x,λ) and â(x,λ), respectively, the formula (12) is replaced by the following formula (13).

â(x,λ)=k_{H}(λ)·d_{H}+k_{E}(λ)·d_{E}+k_{R}(λ)·d_{R} (13)

Since unknown variables in the formula (13) are the three dye amounts d_{H}, d_{E}, and d_{R}, the dye amounts d_{H}, d_{E}, and d_{R} may be obtained by creating and calculating simultaneous equations of the formula (13) for at least three different wavelengths λ. In order to increase the accuracy, simultaneous equations of the formula (13) may be created and calculated for four or more different wavelengths λ, so that multiple regression analysis is performed. For example, when three simultaneous equations of the formula (13) for three wavelengths λ₁, λ₂, and λ₃ are created, the equations may be expressed by matrices as the following formula (14).

$$( \begin{matrix}\hat{a} ( x , {λ}_{1} ) \\ \hat{a} ( x , {λ}_{2} ) \\ \hat{a} ( x , {λ}_{3} )\end{matrix} ) = ( \begin{matrix}{k}_{H} ( {λ}_{1} ) & {k}_{E} ( {λ}_{1} ) & {k}_{R} ( {λ}_{1} ) \\ {k}_{H} ( {λ}_{2} ) & {k}_{E} ( {λ}_{2} ) & {k}_{R} ( {λ}_{2} ) \\ {k}_{H} ( {λ}_{3} ) & {k}_{E} ( {λ}_{3} ) & {k}_{R} ( {λ}_{3} )\end{matrix} ) ( \begin{matrix}{d}_{H} \\ {d}_{E} \\ {d}_{R}\end{matrix} ) \qquad ( 14 )$$

The formula (14) is replaced by the following formula (15).

Â(x)=K₀D₀(x) (15)

When the number of samples in the wavelength direction is represented by m, a matrix Â(x) is a matrix with m row and one column corresponding to â(x,λ), a matrix K₀ is a matrix with m rows and three columns corresponding to the reference dye spectra k(λ), and a matrix D₀(x) is a matrix with three rows and one column corresponding to the dye amounts d_{H}, d_{E}, and d_{R} at a point x in the formula (15).

The dye amounts d_{H}, d_{E}, and d_{R} are calculated by using the least squares method according to the formula (15). The least squares method is a method of estimating the matrix D₀(x) such that a sum of squares of error is smallest in a simple linear regression equation. An estimated value D₀̂(x) of the matrix D₀(x) obtained by the least squares method is given by the following formula (16).

\hat{D}₀(x)=(K₀^{T}K₀)⁻¹K₀^{T}Â(x) (16)

In the formula (16), the estimated value D₀̂(x) is a matrix having the estimated dye amounts as elements. Spectral absorbance a˜(x,λ) restored by substituting the estimated dye amounts d̂_{H}, d̂_{E}, and d̂_{R} into the formula (12) is given by the following formula (17). Note that the symbol a˜ means that a symbol “˜(tilde)” representing a restored value is present over a symbol a.

ã(x,λ)=k_{H}(λ)·\hat{d}_{H}+k_{E}(λ)·\hat{d}_{E}+k_{E}(λ)·\hat{d}_{E} (17)

Thus, estimation error e(λ) in the dye amount estimation is given by the following formula (18) from the estimated spectral absorbance â(x,λ) and the restored spectral absorbance a˜(x,λ).

e(λ)=â(x,λ)−ã(x,λ) (18)

The estimation error e(λ) will hereinafter be referred to as a residual spectrum. The estimated spectral absorbance â(x,λ) may be expressed as in the following formula (19) by using the formulas (17) and (18).

â(x,λ)=k_{H}(λ)·\hat{d}_{H}+k_{E}(λ)·\hat{d}_{E}+k_{E}(λ)·\hat{d}_{R}+e(λ) (19)

Note that, although the Lambert-Beer law formulates attenuation of light transmitted by a translucent object on the assumption that no refraction and no scattering occur, refraction and scattering may occur in an actual stained specimen. Thus, modeling of light attenuation caused by the stained specimen with the Lambert-Beer law alone results in an error associated with the modeling. It is, however, highly difficult and practically unfeasible to build a model involving refraction and scattering in a biological specimen. Thus, addition of a residual spectrum, which is an error in modeling in view of the influence of refraction and scattering, prevents occurrence of unnatural color fluctuation due to a physical model.

In observation of reflected light from the object, since the reflected light is affected by optical factors such as scattering in addition to absorption, the Lambert-Beer law may not be applied to the reflected light as it is. In this case, however, setting appropriate constraint conditions allows estimation of the amounts of dye components in the object based on the Lambert-Beer law.

A case of estimation of the amounts of dye components in a fat region near a mucosa of an organ will be described as an example. FIG. 26 is a set of graphs illustrating relative absorbances (reference spectra) of oxygenated hemoglobin, carotene, and bias. Among the graphs, (b) of FIG. 26 illustrates the same data as in (a) of FIG. 26 with a larger scale on the vertical axis and with a smaller range. In addition, bias is a value representing luminance unevenness in an image, which does not depend on the wavelength.

The amounts of respective dye components are calculated from absorption spectra in a region in which fat is imaged based on the reference spectra of oxygenated hemoglobin, carotene, and bias. In this case, the wavelength band is limited to 460 to 580 nm, in which the absorption characteristics of oxygenated hemoglobin contained in blood, which is dominant in a living body, do not change significantly and the wavelength dependence of scattering has little influence, so that optical factors other than absorption do not have influence, and absorbances within the wavelength band are used to estimate the amounts of dye components.

FIG. 27 is a set of graphs illustrating absorbances (estimated values) restored from the estimated amounts of oxygenated hemoglobin according to the formula (14), and measured values of oxygenated hemoglobin. Among the graphs, (b) of FIG. 27 illustrates the same data as in (a) of FIG. 27 with a larger scale on the vertical axis and with a smaller range. As illustrated in FIG. 27, the measured values and the estimated values are approximately the same within the limited wavelength band of 460 to 580 nm. In this manner, even when reflected light from an object is observed, limiting the wavelength band to a narrow range in which the absorption characteristics of the dye components do not significantly change allows estimation of the amounts of components with high accuracy.

In contrast, at the outside of the limited wavelength band, that is, in wavelength bands lower than 460 nm and higher than 580 nm, the measured values and the estimated values are different from one another and estimation error is observed. This is thought to be because the Lambert-Beer law, which expresses absorption phenomena, may not approximate the values since optical factors such as scattering other than absorption affect the reflected light from the object. Thus, it is generally known that the Lambert-Beer law is not satisfied when reflected light is observed.

In recent years, research on measurement of the depth of specified tissue in a living body based on an image capturing the living body has been carried out. For example, JP 2011-098088 A discloses a technology of acquiring a broadband image data corresponding to broadband light in a wavelength band of 470 to 700 nm, for example, and narrow-band image data corresponding to narrow-band light having a wavelength limited to 445 nm, for example, calculating a luminance ratio of pixels at corresponding positions in the broadband image data and the narrow-band image data, obtaining a blood vessel depth corresponding to the calculated luminance ratio based on correlations between luminance ratios and blood vessel depths obtained in advance by experiments or the like, and determining whether or not the blood vessel depth corresponds to a surface layer.

In addition, WO 2013/115323 A discloses a technology of using a difference in optical characteristics between an adipose layer and tissue surrounding the adipose layer at a specified part so as to form an optical image in which a region of an adipose layer including relatively more nerves than surrounding tissue and a region of the surrounding tissue are distinguished from each other, and displaying distribution or a boundary between the adipose layer and the surrounding tissue based on the optical image. This facilitates recognition of the position of the surface of an organ to be removed in an operation to prevent damage to nerves surrounding the organ.

### SUMMARY

An image processing device for estimating a depth of specified tissue included in an object based on an image obtained by capturing the object with light with a plurality of wavelengths includes: an absorbance calculating unit configured to calculate absorbances at the wavelengths based on pixel values of a plurality of pixels constituting the image; a component amount estimating unit configured to estimate amounts of two or more kinds of light absorbing components contained respectively in two or more kinds of tissue including the specified tissue by using an absorbance in a first wavelength band among the absorbances at the wavelengths calculated by the absorbance calculating unit, the first wavelength band being a part of the wavelengths; an estimation error calculating unit configured to calculate estimation errors caused by the component amount estimating unit; and a depth estimating unit configured to estimate a depth at which the specified tissue is present in the object based on an estimation error in a second wavelength band with shorter wavelength than the first wavelength band among the estimation errors.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a graph illustrating absorption spectra measured based on an image of a specimen taken out from a living body;

FIG. 2 is a set of schematic views illustrating a cross section of a region near a mucosa of a living body;

FIG. 3 is a set of graphs illustrating reference spectra of relative absorbances of oxygenated hemoglobin and carotene;

FIG. 4 is a block diagram illustrating an example configuration of an imaging system according to a first embodiment;

FIG. 5 is a schematic view illustrating an example configuration of an imaging device illustrated in FIG. 4;

FIG. 6 is a flowchart illustrating operation of an image processing device illustrated in FIG. 4;

FIG. 7 is a set of graphs illustrating estimated values and measured values of absorbance in a region in which blood is present near a surface of a mucosa;

FIG. 8 is a set of graphs illustrating estimated values and measured values of absorbance in a region in which blood is present at a depth;

FIG. 9 is a graph illustrating estimation error in the region in which blood is present near the surface of the mucosa and in the region in which blood is present at a depth;

FIG. 10 is a graph illustrating normalized estimation errors in the region in which blood is present near the surface of the mucosa and in the region in which blood is present at a depth;

FIG. 11 is a graph illustrating comparison between normalized estimation error in a region in which a blood layer is present near the surface of a mucosa and normalized estimation error in a region in which a blood layer is present at a depth;

FIG. 12 is a block diagram illustrating an example configuration of an image processing device according to a second embodiment;

FIG. 13 is a flowchart illustrating operation of the image processing device illustrated in FIG. 12;

FIG. 14 is a graph illustrating absorbances (measured values) in a region in which blood is present near the surface of a mucosa and normalized absorbances;

FIG. 15 is a graph illustrating absorbance (measured values) in a region in which blood is present at a depth and normalized absorbances;

FIG. 16 is a set of graphs illustrating estimated values and measured values of normalized absorbance in a region in which blood is present near a surface of a mucosa;

FIG. 17 is a set of graphs illustrating estimated values and measured values of normalized absorbance in a region in which blood is present at a depth;

FIG. 18 is a graph illustrating normalized estimation error in the region in which blood is present near the surface of the mucosa and in the region in which blood is present at a depth;

FIG. 19 is a graph illustrating comparison in normalized estimation error between the region in which blood is present near the surface of the mucosa and the region in which blood is present at a depth;

FIG. 20 is a block diagram illustrating an example configuration of an image processing device according to a third embodiment;

FIG. 21 is a schematic view illustrating an example of display of a region of fat;

FIG. 22 is a set of graphs for explaining the sensitivity characteristics of an imaging device applicable to the first to third embodiments;

FIG. 23 is a block diagram illustrating an example configuration of an image processing device according to a fifth embodiment;

FIG. 24 is a schematic diagram illustrating an example configuration of an imaging system according to a sixth embodiment;

FIG. 25 is a schematic diagram illustrating an example configuration of an imaging system according to a seventh embodiment;

FIG. 26 is a set of graphs illustrating reference spectra of oxygenated hemoglobin, carotene, and bias in a fat region; and

FIG. 27 is a set of graphs illustrating estimated values and measured values of the absorbance of oxygenated hemoglobin.

### DETAILED DESCRIPTION

Embodiments of an image processing device, an image processing method, an image processing program, and an imaging system according to the present disclosure will now be described in detail with reference to the drawings. Note that the present disclosure is not limited to the embodiments. In depiction of the drawings, the same components will be designated by the same reference numerals.

### First Embodiment

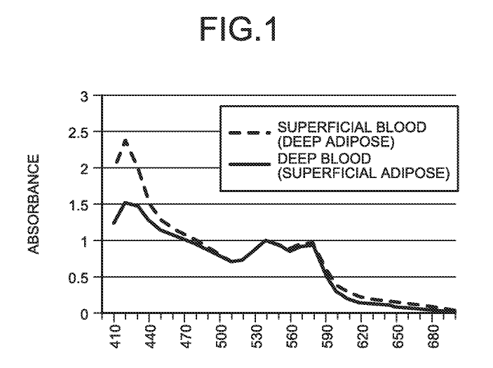

First, the principle of an image processing method according to a first embodiment will be explained with reference to FIGS. 1 to 3. FIG. 1 is a graph illustrating absorption spectra measured based on an image of a specimen taken out from a living body. In FIG. 1, a graph of superficial blood (deep fat) is obtained by measuring five points within a region in which blood is present near the surface of a mucosa and fat is present at a depth, normalizing absorption spectra obtained at the respective points according to absorbance at a wavelength of 540 nm and averaging the normalization result. In contrast, a graph of deep blood (superficial fat) is obtained by measuring five points within a region in which fat is exposed to the surface of a mucosa and blood is present at a depth, and performing similar processing.

As illustrated in FIG. 1, there is a difference in absorbance between the region of superficial blood (deep fat) and the region of deep blood (superficial fat) in a short wavelength band of 400 to 440 nm. In addition, overall, the absorbance is relatively high in the region of superficial blood (deep fat) and relatively low in the region of deep blood (superficial fat).

Typically, light with a short wavelength is likely to be reflected by a tissue surface and light with a long wavelength is likely to reach the depth of tissue, absorbance in a short wavelength band is deemed to reflect superficial tissue and absorbance in a long wavelength band is deemed to reflect deep tissue. Thus, the difference in absorption in the short wavelength band of 400 to 440 nm illustrated in FIG. 1 is thought to be caused by a difference in superficial tissue.

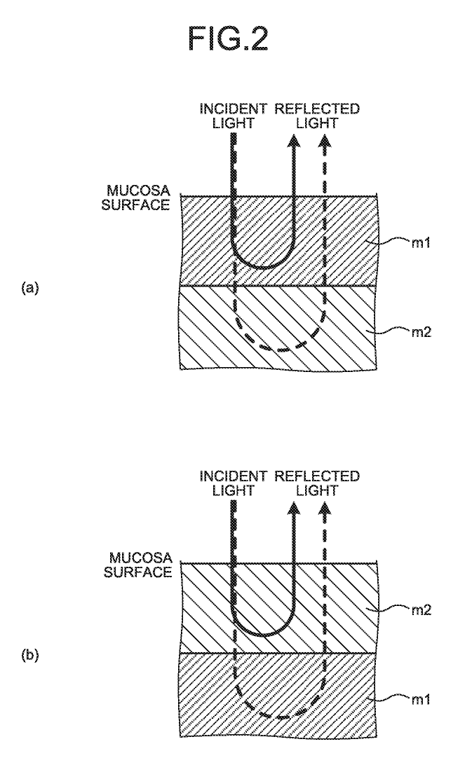

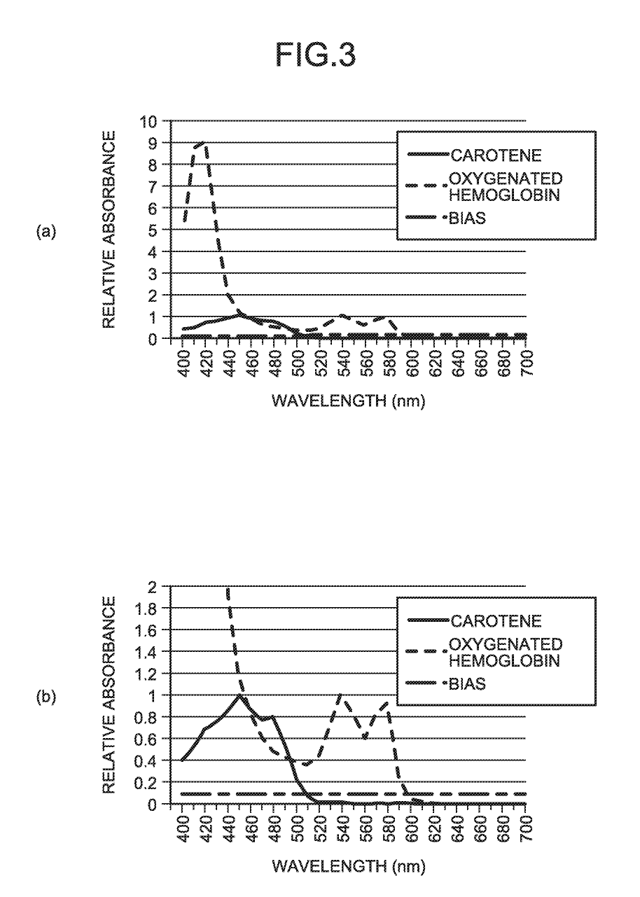

FIG. 2 illustrates schematic views illustrating a cross section of a region near a mucosa of a living body. In FIG. 2, (a) illustrates a region in which a blood layer m1 is present near a mucosa surface and a fat layer m2 is present at a depth. (b) of FIG. 2 illustrates a region in which a fat layer m2 is present near a mucosa surface and a blood layer m1 is present at a depth. In addition, FIG. 3 is a set of graphs illustrating reference spectra of relative absorbances of oxygenated hemoglobin, which is a light absorbing component (pigment) contained in blood, and carotene, which is a light absorbing component contained in fat. Among the graphs, (b) of FIG. 3 illustrates the same data as in (a) of FIG. 3 with a larger scale on the vertical axis and with a smaller range. In addition, bias illustrated in FIG. 3 is a value representing luminance unevenness in an image, which does not depend on the wavelength. In the description below, bias is also regarded as one of light absorbing components in computation.

As illustrated in FIG. 3, oxygenated hemoglobin exhibits strong absorption characteristics in a short wavelength band of 400 to 440 nm. Thus, when a blood layer m1 is present near the surface as illustrated in (a) of FIG. 2, light with a short wavelength is mostly absorbed by the blood present near the surface and slightly reflected. In addition, part of the light with a short wavelength is assumed to reach a depth and not to be absorbed by the fat but to be reflected and returned. Thus, as illustrated in FIG. 1, strong absorption of a wavelength component in the short wavelength band is observed in light reflected in the region of superficial blood (deep fat).

In contrast, as illustrated in FIG. 3, carotene contained in fat does not exhibit so strong absorption characteristics in the short wavelength band of 400 to 440 nm. Thus, when a fat layer m2 is present near the surface as illustrated in (b) of FIG. 2, light with a short wavelength is mostly not absorbed near the surface but is reflected and returned. In addition, part of the light with a short wavelength reaches a depth, where the light is strongly absorbed and slightly reflected. Thus, as illustrated in FIG. 1, in the light reflected in the region of deep blood (superficial fat), absorption of a wavelength component in the short wavelength band is relatively weak.

In contrast, in a medium wavelength band of 460 to 580 nm, absorbance in the region of superficial blood (deep fat) and absorbance in the deep blood (superficial fat) have similar spectral shapes. These spectral shapes are approximate to those of oxygenated hemoglobin illustrated in FIG. 3. Note that light in the medium wavelength band mostly reach a depth of tissue, and is reflected and returned. In addition, blood is also present in both of the region of superficial blood (deep fat) and the region of deep blood (superficial fat). Thus, the reason for which the absorbances in both of the regions are approximate to the spectral shapes of oxygenated hemoglobin is thought to be that light in the medium wavelength band is partially absorbed by the blood layer m1 and the remaining light is returned whether near the surface or at a depth.

Thus, it is possible to determine whether blood is present near the surface or at a depth or whether fat is exposed to the surface or present at a depth (under a membrane) in an observed region by normalizing the spectra in both of the region of superficial blood (deep fat) and the region of deep blood (superficial fat) according to the absorbances in the medium wavelength band whose spectral shapes are approximate to each other, and measuring the difference in absorbance in the short wavelength band in which the spectral shapes in the regions are significantly different from each other.

Note that, since blood is dominant in a living body, the light absorbing component that is typically used for determination on whether the depth of blood is shallow or deep is oxygenated hemoglobin. Since, however, tissue such as fat other than blood is also present in a living body, the influence of light absorbing components contained in tissue other than blood, such as the influence of carotene contained in fat, needs to be excluded before evaluation. For this purpose, the amounts of oxygenated hemoglobin and carotene may be estimated with a model based on the Lambert-Beer law, and the influence of the amounts of components other than oxygenated hemoglobin contained in blood, that is, the amount of carotene contained in fat may be excluded before evaluation.

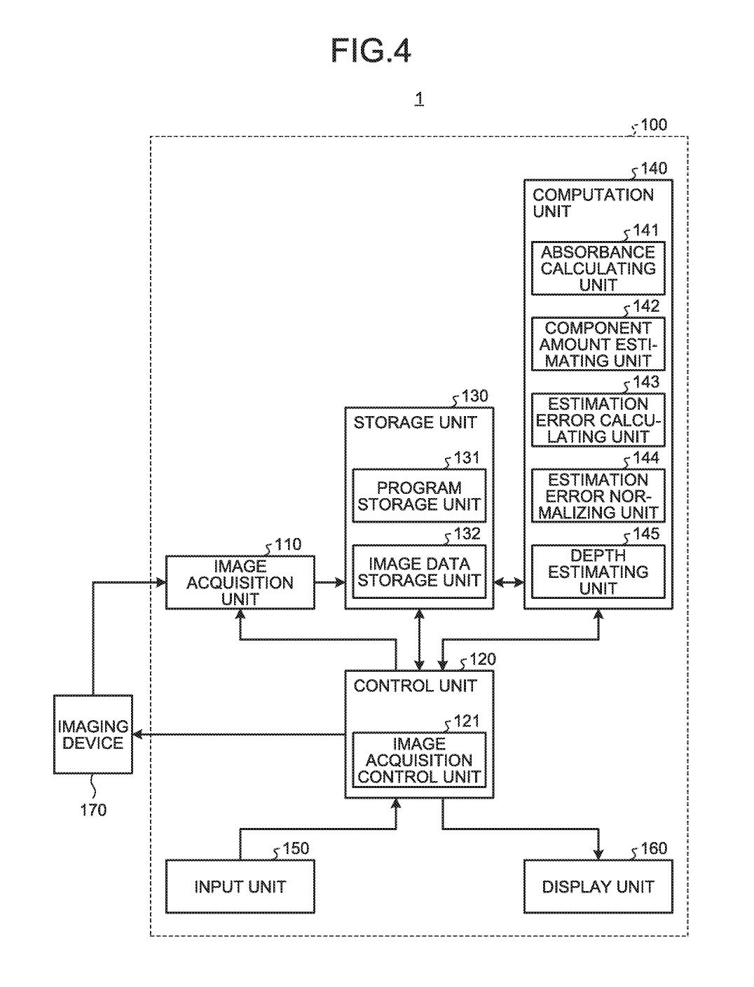

FIG. 4 is a block diagram illustrating an example configuration of an imaging system according to the first embodiment. As illustrated in FIG. 4, the imaging system 1 according to the present embodiment includes an imaging device 170 such as a camera, and an image processing device 100 constituted by a computer such as a personal computer connectable with the imaging device 170.

The image processing device 100 includes an image acquisition unit 110 for acquiring image data from the imaging device 170, a control unit 120 for controlling overall operation of the system including the image processing device 100 and the imaging device 170, a storage unit 130 for storing image data and the like acquired by the image acquisition unit 110, a computation uni 140 for performing predetermined image processing based on the image data stored in the storage unit 130, an input unit 150, and a display unit 160.

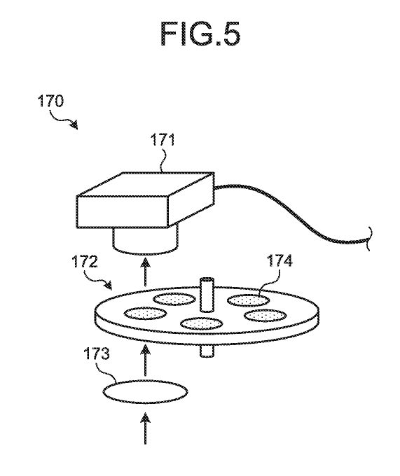

FIG. 5 is a schematic view illustrating an example configuration of the imaging device 170 illustrated in FIG. 4. The imaging device 170 illustrated in FIG. 5 includes a monochromatic camera 171 for generating image data by converting received light into electrical signals, a filter unit 172, and a tube lens 173. The filter unit 172 includes a plurality of optical filters 174 having different spectral characteristics, and switches between optical filters 174 arranged on an optical path of light incident on the monochromatic camera 171 by rotating a wheel. For capturing a multiband image, the optical filters 174 having different spectral characteristics are sequentially positioned on the optical path, and an operation of causing reflected light from an object to form an image on a light receiving surface of the monochromatic camera 171 via the tube lens 173 and the filter unit 172 is repeated for each of the filters 174. Note that the filter unit 172 may be provided on the side of an illumination device for irradiating an object instead of the side of the monochromatic camera 171.

In addition, a multiband image may be acquired in such a manner that an object is irradiated with light having different wavelengths in respective bands. In addition, the number of bands of a multiband image is not limited as long as the number is not smaller than the number of kinds of light absorbing components contained in an object. For example, the number of bands may be three such that an RGB image is acquired.

Alternatively, liquid crystal tunable filter or an acousto-optic tunable filter capable of changing spectral characteristics may be used instead of the optical filters 174 having different spectral characteristics. In addition, a multiband image may be acquired in such a manner that a plurality of light beams having different spectral characteristics may be switched to irradiate an object.

With reference back to FIG. 4, the image acquisition unit 110 has an appropriate configuration depending on the mode of the system including the image processing device 100. For example, when the imaging device 170 illustrated in FIG. 5 is connected to the image processing device 100, the image acquisition unit 110 is constituted by an interface for reading image data output from the imaging device 170. Alternatively, when a server for saving image data generated by the imaging device 170 is provided, the image acquisition unit 110 is constituted by a communication device or the like connected with the server, and acquires image data through data communication with the server. Alternatively, the image acquisition unit 110 may be constituted by a reader, on which a portable recording medium is removably mounted, for reading out image data recorded on the recording medium.

The control unit 120 is constituted by a general-purpose processor such as a central processing unit (CPU) or a special-purpose processor such as various computation circuits configured to perform specific functions such as an application specific integrated circuit (ASIC). When the control unit 120 is a general-purpose processor, the control unit 120 performs providing, instructions, transferring data, and the like to respective components of the image processing device 100 by reading various programs stored in the storage unit 130 to generally control the overall operation of the image processing device 100. When the control unit 120 is a special-purpose processor, the processor may perform various processes alone or may use various data and the like stored in the storage unit 130 so that the processor and the storage unit 130 perform various processes in cooperation or in combination.

The control unit 120 includes an image acquisition control unit 121 for controlling operation of the image acquisition unit 110 and the imaging device 170 to acquire an image, and controls the operation of the image acquisition unit 110 and the imaging device 170 based on an input signal input from the input unit 150, an image input from the image acquisition unit 110, and a program, data, and the like stored in the storage unit 130.

The storage unit 130 is constituted by various IC memories such as a read only memory (ROM) or a random access memory (RAM) such as an updatable flash memory, an information storage device such as a hard disk or a CD-ROM that is built in or connected via a data communication terminal, a writing/reading device that reads/writes information from/to the information storage device, and the like. The storage unit 130 includes a program storage unit 131 for storing image processing programs, and an image data storage unit 132 for storing image data, various parameters, and the like to be used during execution of the image processing programs.

The computation unit 140 is constituted by a general-purpose processor such as a CPU or a special-purpose processor such as various computation circuits for performing specific functions such as an ASIC. When the computation unit 140 is a general-purpose processor, the processor reads an image processing program stored in the program storage unit 131 so as to perform image processing of estimating a depth at which specified tissue is present based on a multiband image. Alternatively, when the computation unit 140 is a special-purpose processor, the processor may perform various processes alone or may use various data and the like stored in the storage unit 130 so that the processor and the storage unit 130 perform image processing in cooperation or in combination.

More specifically, the computation unit 140 includes an absorbance calculating unit 141 for calculating absorbance in an object based on an image acquired by the image acquisition unit 110, a component amount estimating unit 142 for estimating the amounts of two or more kinds of light absorbing components present in the object based on the absorbance, an estimation error calculating unit 143 for calculating estimation error, an estimation error normalizing unit 144 for normalizing the estimation error, and a depth estimating unit 145 for estimating the depth of specified tissue based on the normalized estimation error.

The input unit 150 is constituted by input devices such as a keyboard, a mouse, a touch panel, and various switches, for example, and outputs, to the control unit 120, input signals in response to operational inputs.

The display unit 160 is constituted by a display device such as a liquid crystal display (LCD), an electroluminescence (EL) display, or a cathode ray tube (CRT) display, and displays various screens based on display signals input from the control unit 120.

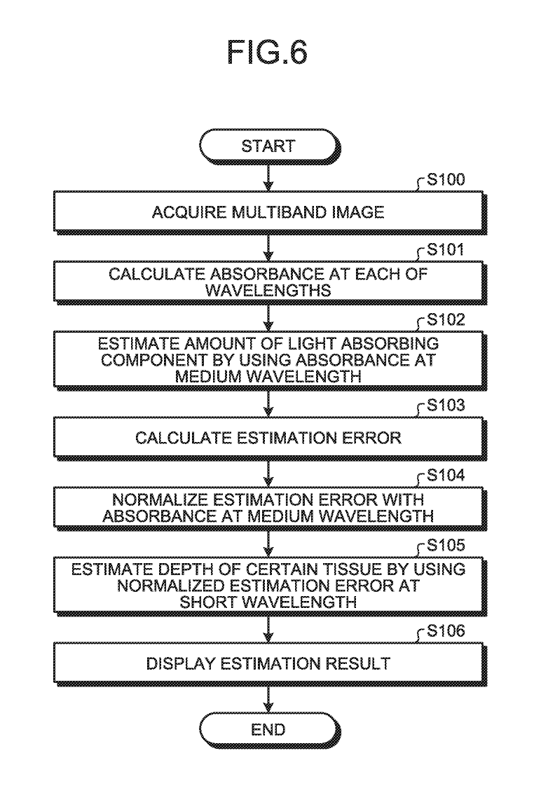

FIG. 6 is a flowchart illustrating operation of the image processing device 100. First, in step S100, the image processing device 100 causes the imaging device 170 to operate under the control of the image acquisition control unit 121 to acquire a multiband image obtained by capturing an object with light with plurality of wavelengths. In the first embodiment, multiband imaging in which the wavelength is sequentially shifted by 10 nm between 400 and 700 nm is performed. The image acquisition unit 110 acquires image data of the multiband image generated by the imaging device 170, and stores the image data in the image data storage unit 132. The computation unit 140 acquires the multiband image by reading the image data from the image data storage unit 132.

In subsequent step S101, the absorbance calculating unit 141 obtains pixel values of a plurality of pixels constituting the multiband image, and calculates absorbance at each of the wavelengths based on the pixel values. Specifically, the value of a logarithm of a pixel value in a band corresponding to each wavelength λ is assumed to be an absorbance a(λ) at the wavelength.

In subsequent step S102, the component amount estimating unit 142 estimates the amounts of light absorbing components in the object by using the absorbance at a medium wavelength among the absorbances calculated in step S101. The absorbance at a medium wavelength is used here because the medium wavelength is in a wavelength band in which the light absorption characteristics (see FIG. 3) of oxygenated hemoglobin, which is a light absorbing component contained in blood, which is major tissue of a living body, are stable. Note that the wavelength band in which the light absorption characteristics are stable refers to a wavelength band in which a change in the light absorption characteristics with a change in the wavelength is small, that is, a wavelength band in which the amount of change in the light absorption characteristics between adjacent wavelengths is not larger than a threshold. In the case of oxygenated hemoglobin, such a wavelength band corresponds to 460 to 580 nm.

When a matrix with three rows and one column having the amount d₁ of oxygenated hemoglobin, the amount d₂ of carotene, which is a light absorbing component in fat, and bias d_{bias} as elements is represented by a matrix D, the matrix D is given by the following formula (20). The bias d_{bias} is a value representing luminance unevenness in an image, which does not depend on the wavelength.

D=(K^{T}K)¹K^{T}A (20)

In the formula (20), a matrix A is a matrix with m rows and one column having absorbances a(λ) at m wavelengths λ included in the medium wavelength band as elements. In addition, a matrix K is a matrix with m rows and three columns having a reference spectrum k₁(λ) of oxygenated hemoglobin, a reference spectrum k₂(λ) of carotene, and a reference spectrum k_{bias}(λ) of bias.

In subsequent step S103, the estimation error calculating unit 143 calculates estimation error by using the absorbances a(λ), the amounts d₁ and d₂ of the respective light absorbing components, and the bias d_{bias}. More specifically, restored absorbances a˜(λ) are first calculated with use of the amounts d₁ and d₂ of the respective light absorbing components and the bias d_{bias} by formula (21), and estimation error e(λ) is further calculated from the restored absorbances a˜(λ) and the original absorbances a(λ) by formula (22).

ã(λ)=k₁(λ)·d₁+k₂(λ)·d₁+k_{bias}(λ)·d_{bias} (21)

e(λ)=a(λ)−ã(λ) (22)

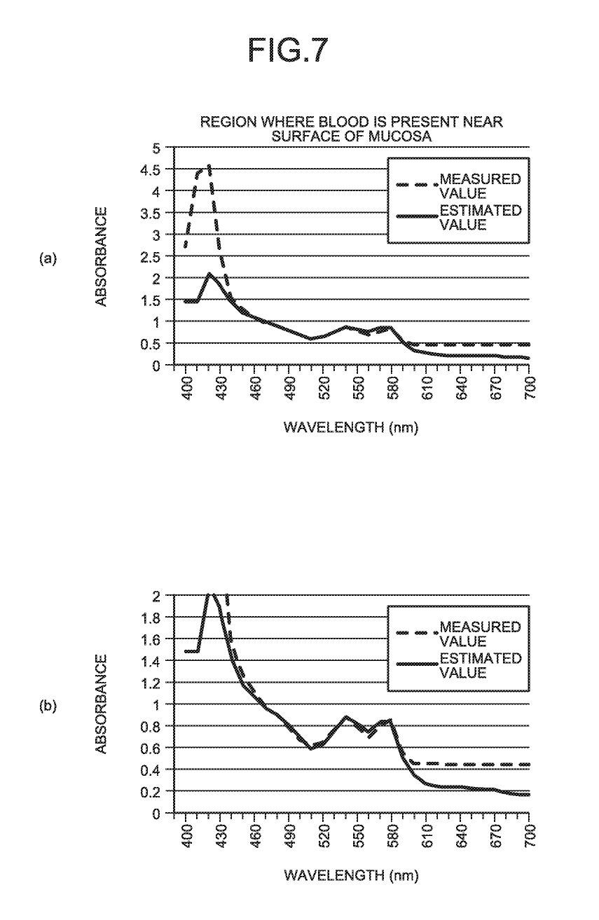

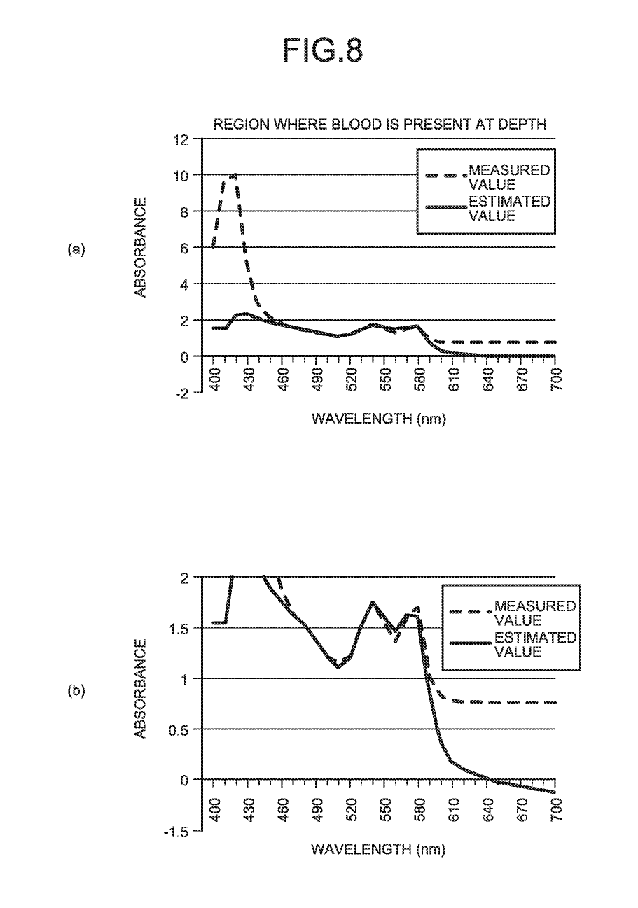

FIG. 7 is a set of graphs illustrating estimated values and measured values of absorbance in a region in which blood is present near a surface of a mucosa. FIG. 8 is a set of graphs illustrating estimated values and measured values of absorbance in a region in which blood is present at a depth. The estimated values of absorbance illustrated in FIGS. 7 and 8 are the absorbances a˜(λ) restored by the formula (21) with use of the amounts of components estimated in step S102. Note that, in FIG. 7, (b) of FIG. 7 illustrates the same data as in (a) of FIG. 7 with a larger scale on the vertical axis and with a smaller range. The same applies to FIG. 8.

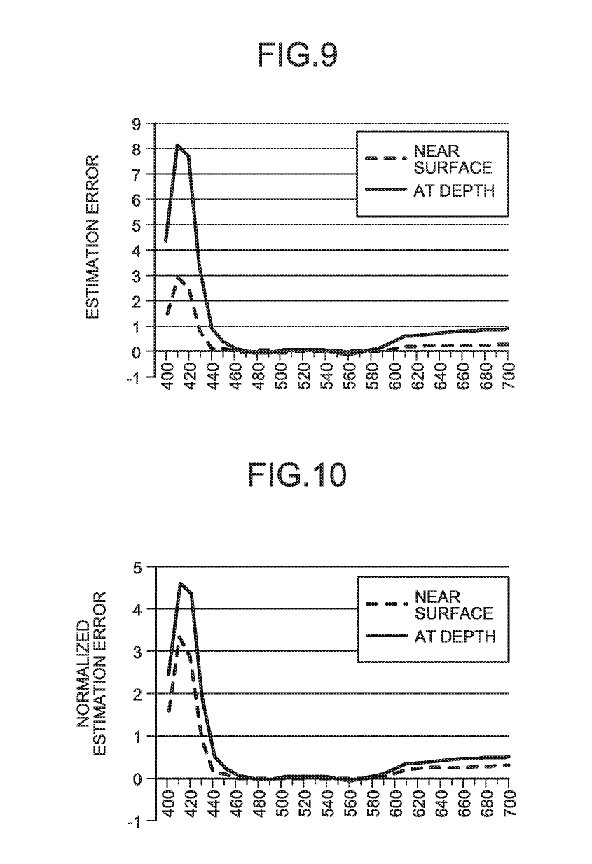

FIG. 9 is a graph illustrating estimation error e(λ) in the region in which blood is present near the surface of the mucosa and in the region in which blood is present at a depth.

In subsequent step S104, the estimation error normalizing unit 144 normalizes the estimation error e(λ) by using the absorbance a(λₘ) at a wavelength λₘ selected from the medium wavelength band (460 to 580 nm) in which the light absorption characteristics of oxygenated hemoglobin are stable among the absorbances calculated in step S101. The normalized estimation error e′(λ) is given by the following formula (23).

$${e}^{′} ( λ ) = \frac{e ( λ )}{a ( {λ}_{m} )} \qquad ( 23 )$$

Alternatively, an average value of the absorbances at respective wavelengths included in the medium wavelength band may be used instead of the absorbance a(λₘ) at the selected wavelength λₘ.

FIG. 10 is a graph illustrating normalized estimation errors e′(λ) obtained by normalizing the estimation errors e(λ) calculated for the region in which blood is present near the surface of the mucosa and the region in which blood is present at a depth according to the absorbance at 540 nm.

In subsequent step S105, the depth estimating unit 145 estimates the depth of the blood layer as specified tissue by using a normalized estimation error e′(λₛ) at a short wavelength among the normalized estimation errors e′(λ). Note that a short wavelength refers to a wavelength shorter than the medium wavelength band in which the light absorption characteristics of oxygenated hemoglobin (see FIG. 3) are stable, and is specifically selected from a band of 400 to 440 nm.

More specifically, the depth estimating unit 145 first calculates an evaluation function Eₑ for estimating the depth of the blood layer by the following formula (24).

Eₑ=Tₑ−e′(λₛ) (24)

In the formula (24), a wavelength λₛ is any one wavelength in the short wavelength band. An average value of normalized estimation errors e′(λₛ) at respective wavelengths included in the short wavelength may be used instead of normalized estimation error e′(λₛ) at any wavelength λₛ. In addition, a symbol Tₑ represents a threshold that is preset based on experiments or the like and stored in the storage unit 130.

When the evaluation function Eₑ is zero or positive, that is, when the normalized estimation error e′(λₛ) is not larger than a threshold Tₑ, the depth estimating unit 145 determines that the blood layer m1 is present near the surface of a mucosa. In contrast, when the evaluation function Eₑ is negative, that is, when the normalized estimation error e′(λₛ) is larger than the threshold Tₑ, the depth estimating unit 145 determines that the blood layer m1 is present at a depth.

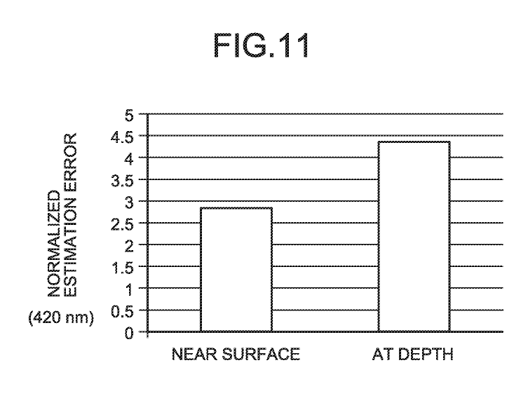

FIG. 11 is a graph illustrating comparison between normalized estimation error e′(λₛ) in the region in which the blood layer m1 is present near the surface of the mucosa and normalized estimation error e′(λₛ) in the region in which the blood layer m1 is present at a depth. In FIG. 11, the values of normalized estimation error e′(420 nm) at 420 nm are illustrated. As illustrated in FIG. 11, there is a significant difference between the case where the blood layer m1 is present near the surface of the mucosa and the case where the blood layer m1 is present at a depth, the values are larger in the latter than in the former.

In subsequent step S106, the computation unit 140 outputs an estimation result, and the control unit 120 displays the estimation result on the display unit 160. The mode in which the estimation result is displayed is not particularly limited. For example, different false colors or hatching of different patterns may be applied on the region in which the blood layer m1 is estimated to be present near the surface of the mucosa (see (a) of FIG. 2) and the region in which the blood layer m1 is estimated to be present at a depth (see (b) of FIG. 2), and displayed on the display unit 160. Alternatively, contour lines of different colors may be superimposed on these regions. Furthermore, highlighting may be applied in such a manner that the luminance of a false color or hatching may be increased or caused to blink so that either one of these region is more conspicuous than the other.

As described above, according to the first embodiment, even though a plurality of kinds of tissue are present in a living body that is an object, a depth at which blood, which is dominant in a living body, is present is estimated with high accuracy.

While the depth of blood is estimated through estimation of the amounts of two kinds of light absorbing components respectively contained in two kinds of tissue, which are blood and fat, in the first embodiment described above, three or more kinds of light absorbing components may be used. For example, for skin analysis, the amounts of three kinds of light absorbing components, which are hemoglobin, melanin, and bilirubin, contained in tissue near the skin may be estimated. Note that hemoglobin and melanin are major pigments constituting the color of the skin, and bilirubin is a pigment appearing as a symptom of jaundice.

### Second Embodiment

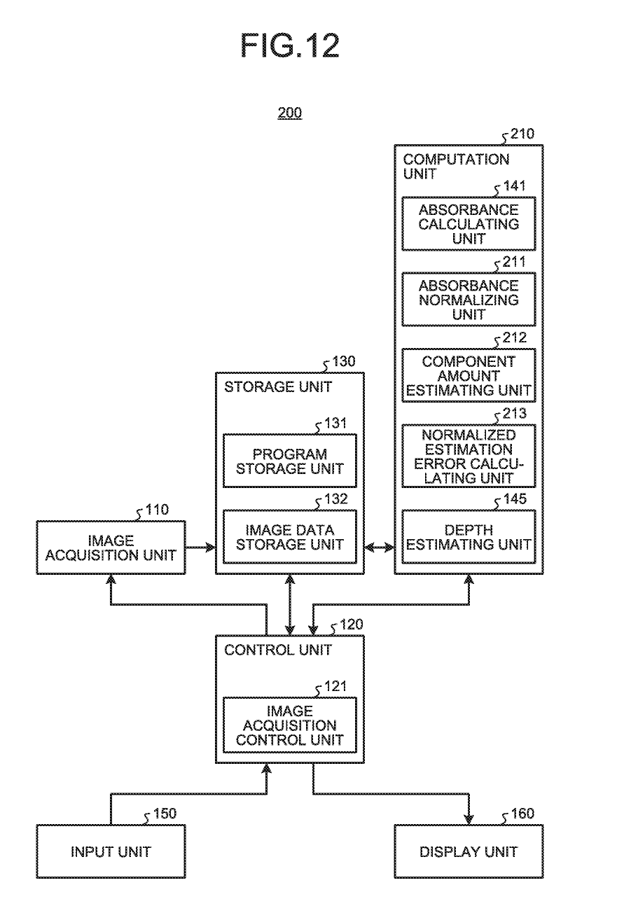

Next, a second embodiment will be described. FIG. 12 is a block diagram illustrating an example configuration of an image processing device according to the second embodiment. As illustrated in FIG. 12, an image processing device 200 according to the second embodiment includes a computation unit 210 instead of the computation unit 140 illustrated in FIG. 4. The configurations and the operations of the respective components of the image processing device 200 other than the computation unit 210 are similar to those in the first embodiment. In addition, the configuration of an imaging device from which the image processing device 200 acquires an image is also similar to that in the first embodiment.

The computation unit 210 normalizes absorbances calculated from respective pixel values of a plurality of pixels constituting a multiband image, estimates the amounts of light absorbing components based on the normalized absorbances, and estimates the depth of tissue based on the component amounts. More specifically, the computation unit 210 includes an absorbance calculating unit 141 for calculating absorbance in an object acquired by the image acquisition unit 110, an absorbance normalizing unit 211 for normalizing the absorbance, a component amount estimating unit 212 for estimating the respective amounts of two or more kinds of light absorbing components present in the object based on the normalized absorbance, a normalized estimation error calculating unit 213 for calculating estimation error, and a depth estimating unit 145 for estimating the depth of specified tissue based on the normalized estimation error. Among these components, the operations of the absorbance calculating unit 141 and the depth estimating unit 145 are similar to those in the first embodiment.

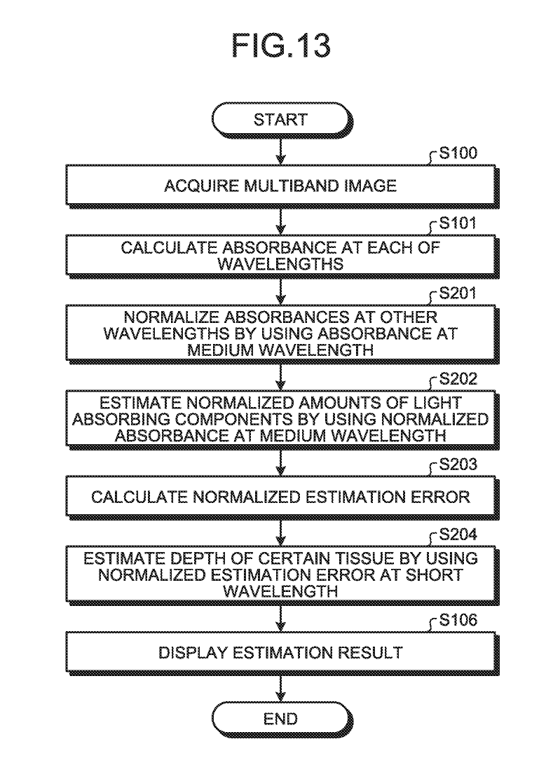

Next, operation of the image processing device 200 will be described. FIG. 13 is a flowchart illustrating operation of the image processing device 200. Note that steps S100 and S101 in FIG. 13 are the same as those in the first embodiment (see FIG. 6).

In step S201 subsequent to step S101, the absorbance normalizing unit 211 uses an absorbance at a medium wavelength among the absorbances calculated in step S101 to normalize absorbances at other wavelengths. In the second embodiment, similarly to the first embodiment, a band of 460 to 580 nm corresponds to medium wavelengths, and an absorbance at a wavelength selected from this band is used in step S201. Hereinafter, absorbance that is normalized is simply referred to as normalized absorbance.

Normalized absorbances a′(λ) are given by the following formula (25) by using the absorbances a(λ) at respective wavelengths λ and the absorbance a(λₘ) at the selected medium wavelength λₘ.

$${a}^{′} ( λ ) = \frac{a ( λ )}{a ( {λ}_{m} )} \qquad ( 25 )$$

Alternatively, an average value of the absorbances at respective wavelengths included in the medium wavelength band may be used instead of the absorbance a(λₘ) at the selected wavelength λₘ for calculation of normalized absorbances a′(λ).

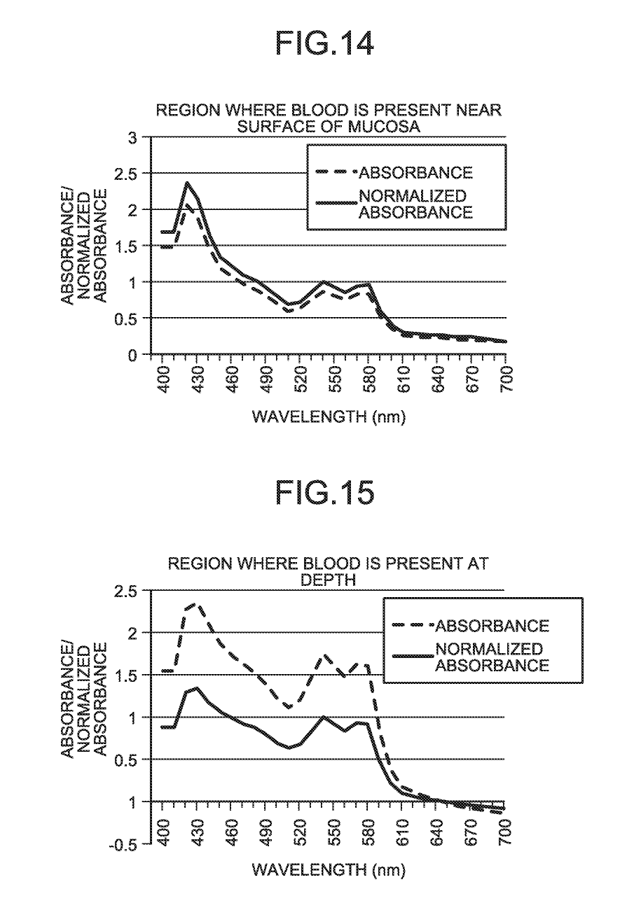

FIG. 14 is a graph illustrating absorbances (measured values) in a region in which blood is present near the surface of a mucosa and normalized absorbances. FIG. 15 is a graph illustrating absorbances (measured values) in a region in which blood is present at a depth and normalized absorbances. In both of FIGS. 14 and 15, other absorbances are normalized with the absorbance at 540 nm.

In subsequent step S202, the component amount estimating unit 212 estimates the normalized amounts of two or more kinds of light absorbing components present in the object by using the normalized absorbance at a medium wavelength among the normalized absorbances calculated in step S201. Similarly to step S201, a band of 460 to 580 nm is used as medium wavelengths.

More specifically, a matrix D′ with three rows and one column having the normalized amount d₁′ of oxygenated hemoglobin, the normalized amount d₂′ of carotene, and normalized bias d_{bias}′ as elements is given by the following formula (26). The normalized bias d_{bias}′ is a value obtained by normalizing a value representing luminance unevenness in an image, which does not depend on the wavelength.

D′=(K^{T}K)⁻¹K^{T}A′ (26)

In the formula (26), a matrix A′ is a matrix with m rows and one column having normalized absorbances a′(λ) at m wavelengths λ included in the medium wavelength band as elements. In addition, a matrix K is a matrix with m rows and three columns having a reference spectrum k₁(λ) of oxygenated hemoglobin, a reference spectrum k₂(λ) of carotene, and a reference spectrum k_{bias}(λ) of bias.

In subsequent step S203, the normalized estimation error calculating unit 213 calculates normalized estimation error e′(λ) by using the normalized absorbances a′(λ), the normalized amounts d₁′ and d₂′ of the respective light absorbing components, and the normalized bias d_{bias}′. Hereinafter, estimation error that is normalized is simply referred to as normalized estimation error. More specifically, restored normalized absorbances a′˜(λ) are first calculated with use of the normalized amounts d₁′ and d₂′ and the normalized bias d_{bias}′ by formula (27), and normalized estimation error e′(λ) is further calculated from the restored normalized absorbances a′˜(λ) and the original normalized absorbances a′(λ) by formula (28).

ã′(λ)=k₁(λ)·d₁′+k₂(λ)·d₂′+k_{bias}(λ)·d_{bias}′ (27)

e′(λ)=a′(λ)−ã(λ) (28)

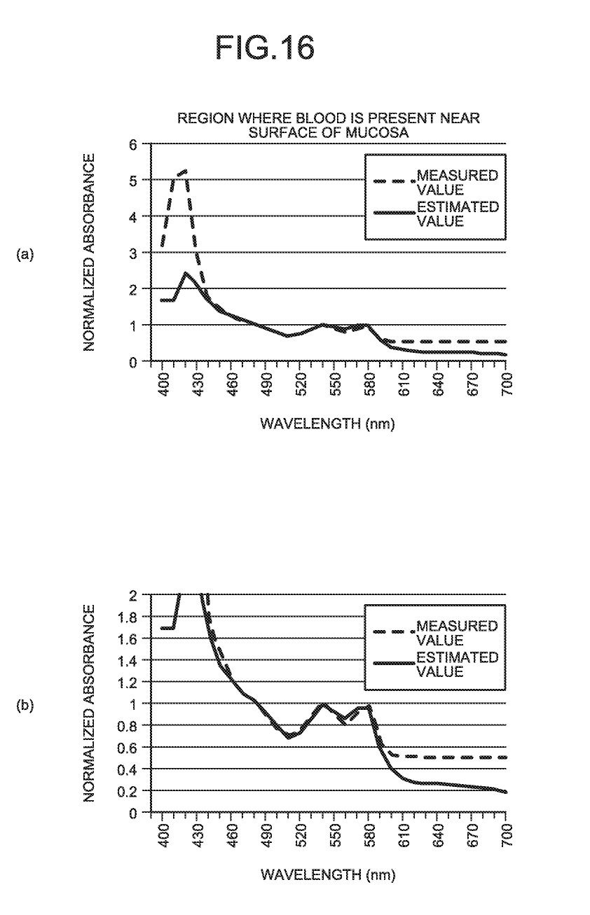

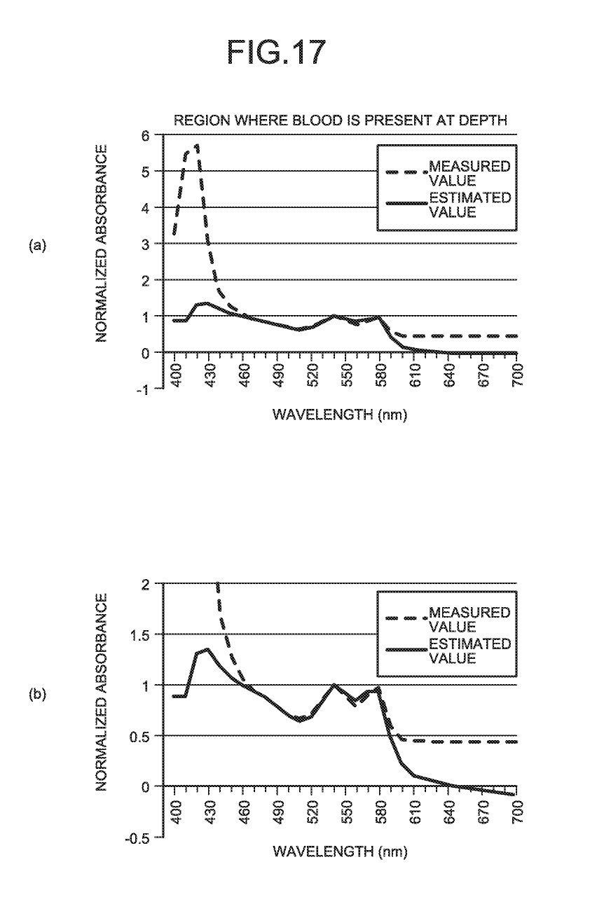

FIG. 16 is a set of graphs illustrating estimated values and measured values of normalized absorbance in a region in which blood is present near a surface of a mucosa. FIG. 17 is a set of graphs illustrating estimated values and measured values of normalized absorbance in a region in which blood is present at a depth. The estimated values of normalized absorbance illustrated in FIGS. 16 and 17 are restored by the formula (27) with use of the normalized amounts of components estimated in step S202. Note that, in FIG. 16, (b) of FIG. 16 illustrates the same data as in (a) of FIG. 16 with a larger scale on the vertical axis and with a smaller range. The same applies to FIG. 17.

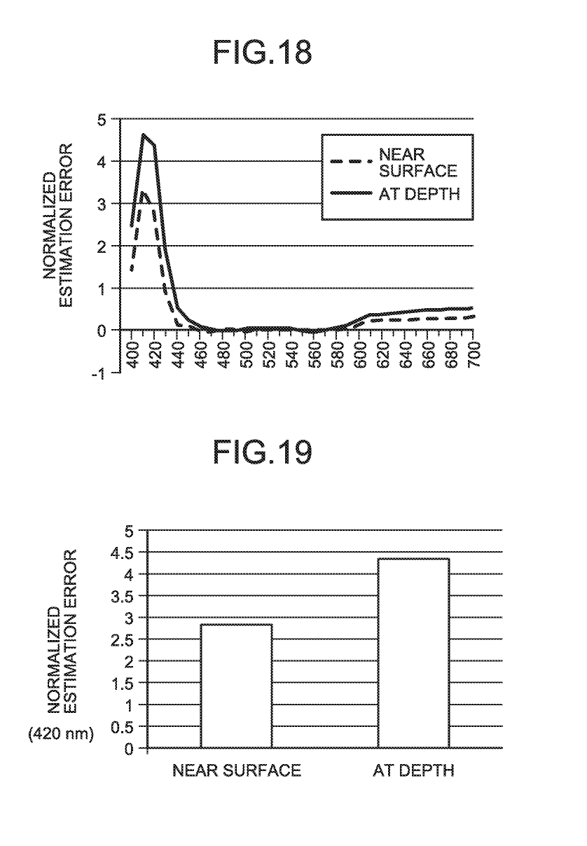

FIG. 18 is a graph illustrating normalized estimation errors e′(λ) in the region in which blood is present near the surface of the mucosa and in the region in which blood is present at a depth.

In subsequent step S204, the depth estimating unit 145 estimates the depth of the blood layer m1 as the specified tissue by using a normalized estimation error e′(λₛ) at a short wavelength among the normalized estimation errors e′(λ). In the second embodiment, similarly to the first embodiment, a band of 400 to 440 nm corresponds to short wavelengths, and normalized estimation error e′(λₛ) at a wavelength selected from this band is used.

FIG. 19 is a graph illustrating comparison in normalized estimation error e′(λₛ) between the region in which blood is present near the surface of the mucosa and the region in which blood is present at a depth. In FIG. 19, the values of normalized estimation error e′(420 nm) at 420 nm are illustrated. As illustrated in FIG. 19, there is a significant difference between the case where the blood layer m1 is present near the surface of the mucosa and the case where the blood layer m1 is present at a depth, the values are larger in the latter than in the former.

Estimation of the depth is performed by calculating the evaluation function Eₑ by the formula (24) and determining whether the evaluation function Eₑ is positive or negative, similarly to the first embodiment (see FIG. 6). Specifically, when the evaluation function Eₑ is positive, the blood layer m1 is determined to be present near the surface of the mucosa, and when the evaluation function Eₑ is negative, the blood layer m1 is determined to be present at a depth. Subsequent step S106 is the same as that in the first embodiment.

As described above, according to the second embodiment, the depths of blood and fat are estimated with high accuracy from the normalized absorbances based on the absorbance of oxygenated hemoglobin contained in blood, which is dominant in a living body.

In the second embodiment as well, the amounts of three or more kinds of light absorbing components may be estimated for estimation of the depth of specified tissue.

### Third Embodiment

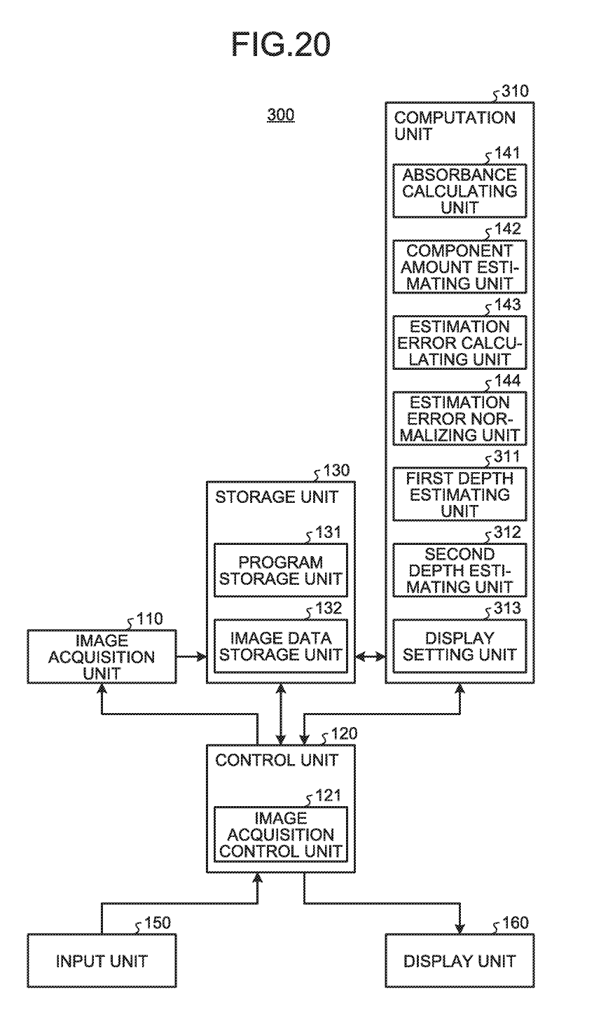

Next, a third embodiment will be described. FIG. 20 is a block diagram illustrating an example configuration of an image processing device according to the third embodiment. As illustrated in FIG. 20, an image processing device 300 according to the third embodiment includes a computation unit 310 instead of the computation unit 140 illustrated in FIG. 4. The configurations and the operations of the respective components of the image processing device 300 other than the computation unit 310 are similar to those in the first embodiment. In addition, the configuration of an imaging device from which the image processing device 300 acquires an image is also similar to that in the first embodiment.

Note that fat observed in a living body includes fat exposed to the surface of a mucosa (exposed fat) and fat that is covered by a mucosa and may be seen therethrough (submucosal fat). In terms of operative procedures, submucosal fat is important. This is because exposed fat may be easily seen with eyes. Thus, technologies for such display that allows operators to easily recognize submucosal fat have been desired. In the third embodiment, the depth of fat is estimated based on the depth of blood, which is major tissue in a living body so that recognition of the submucosal fat is facilitated.

The computation unit 310 includes a first depth estimating unit 311, a second depth estimating unit 312, and a display setting unit 313 instead of the depth estimating unit 145 illustrated in FIG. 4. The operations of the absorbance calculating unit 141, the component amount estimating unit 142, the estimation error calculating unit 143, and the estimation error normalizing unit 144 are similar to those in the first embodiment.

The first depth estimating unit 311 estimates the depth of blood, which is major tissue in a living body, based on the normalized estimation errors e′(λₛ) calculated by the estimation error normalizing unit 144. The method for estimating the depth of blood is similar to that in the first embodiment (see step S105 in FIG. 6).

The second depth estimating unit 312 estimates the depth of tissue other than blood, that is specifically fat, based on the result of estimation by the first depth estimating unit 311. Note that two or more kinds of tissue have a layered structure in a living body. For example, a mucosa in a living body has a region in which a blood layer m1 is present near the surface and a fat layer m2 is present at a depth as illustrated in (a) of FIG. 2, or a region in which a fat layer m2 is present near the surface and a blood layer m1 is present at a depth as illustrated in (b) of FIG. 2.

Thus, when a blood layer m1 is estimated to be present near the surface by the first depth estimating unit 311, the second depth estimating unit 312 estimates that a fat layer m2 is present at a depth. Conversely, when a blood layer m1 is estimated to be present at a depth by the first depth estimating unit 311, the second depth estimating unit 312 estimates that a fat layer m2 is present near the surface. Estimation of the depth of blood, which is major tissue in a living body, in this manner allows estimation of the depth of other tissue such as fat.

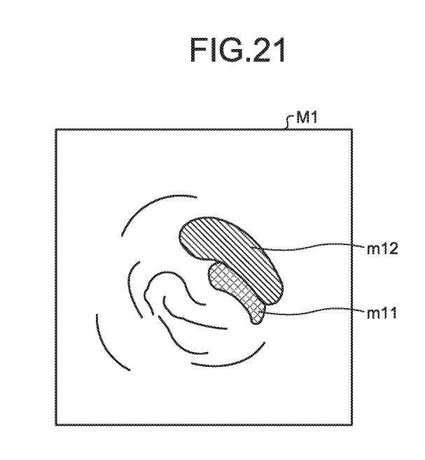

The display setting unit 313 sets a display mode of a region of fat in an image to be displayed on the display unit 160 according to the depth estimation result from the second depth estimating unit 312. FIG. 21 is a schematic view illustrating an example of display of a region of fat. As illustrated in FIG. 21, the display setting unit 313 sets, in an image M1, different display modes for a region m11 in which blood is estimated to be present near the surface of a mucosa and fat is estimated to be present at a depth and a region m12 in which blood is estimated to be present at a depth and fat is estimated to be present near the surface. In this case, the control unit 120 displays the image M1 on the display unit 160 according to the display mode set by the display setting unit 313.

Specifically, when false colors or hatching is applied to all the regions in which fat is present, different colors or patterns are used for the region m11 in which fat is present at a depth and the region m12 in which fat is exposed to the surface. Alternatively, only either one of the region m11 in which fat is present at a depth and the region m12 in which fat is exposed to the surface may be colored. Still alternatively, the signal value of an image signal for display may be adjusted so that the false color changes depending on the amount of fat instead of uniform application of a false color.

In addition, contour lines of different colors may be superimposed on the regions m11 and m12. Furthermore, highlighting may be applied in such a manner that the false color or the contour line in either of the regions m11 and m12 is caused to blink or the like.

Such a display mode of the regions m11 and m12 may be appropriately set according to the purpose of observation. For example, when an operation to remove an organ such as a prostate, there is a demand for facilitating recognition of the position of fat in which many nerves are present. Thus, in this case, the region m11 in which the fat layer m2 is present at a depth is preferably displayed in a more highlighted manner.

As described above, according to the third embodiment, since the depth of blood, which is major tissue in a living body, is estimated and the depth of other tissue such as fat is estimated based on the relation with the major tissue, the depth of tissue other than major tissue may also be estimated in a region in which two or more kinds of tissue are layered.

In addition, according to the third embodiment, since the display mode in which the regions are displayed is changed depending on the positional relation of blood and fat, a viewer of the image is capable of recognize the depth of tissue of interest more clearly.

While the normalized estimation error is calculated in the same manner as in the first embodiment and the depth of blood is estimated based on the normalized estimation error in the third embodiment described above, the normalized estimation error may be calculated in the same manner as in the second embodiment.

### Fourth Embodiment

Next, a fourth embodiment is described. While it is assumed that multispectral imaging is performed in the first to third embodiments described above, imaging with any three wavelengths is sufficient for estimation of three values, which are the amounts of two components and the bias.

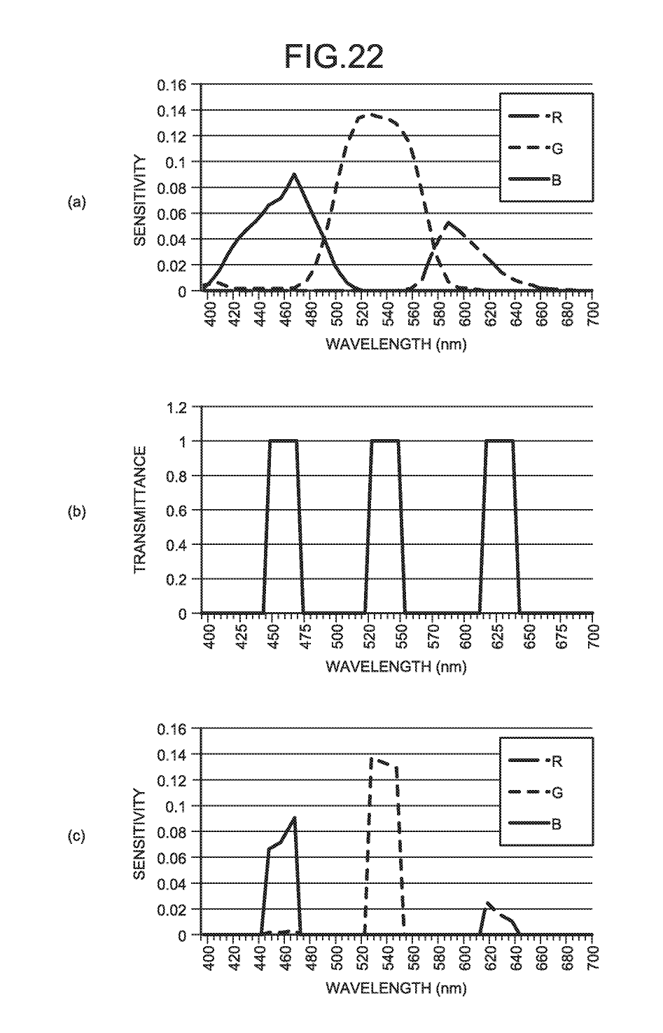

In this case, the imaging device 170 from which the image processing devices 100, 200, and 300 obtain an image may have a configuration including an RGB camera with a narrow-band filter. FIG. 22 illustrates graphs for explaining the sensitivity characteristics of such an imaging device. (a) of FIG. 22 illustrates the sensitivity characteristics of an RGB camera, (b) of FIG. 22 illustrates the transmittance of a narrow-band filter, and (c) of FIG. 22 illustrates the total sensitivity characteristics of the imaging device.

When an object image is formed with RGB via the narrow-band filter, the total sensitivity characteristics of the imaging device are given by a product (see (c) of FIG. 22) of the sensitivity characteristics of the camera (see (a) of FIG. 22) and the sensitivity characteristics of the narrow-band filter (see (b) of FIG. 22).

### Fifth Embodiment

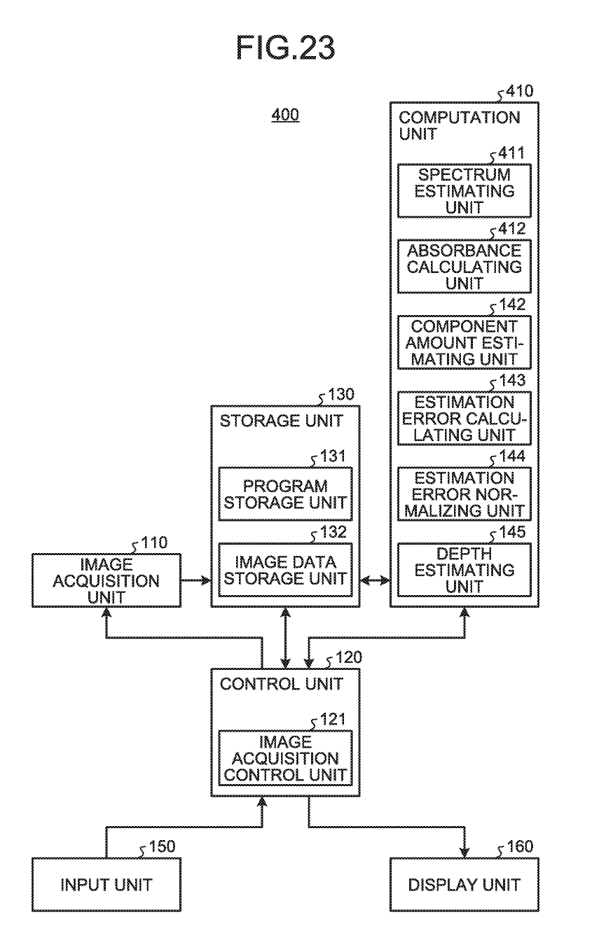

Next, a fifth embodiment is described. While it is assumed that multispectral imaging is performed in the first to third embodiments, an optical spectrum may be estimated with use of a small number of bands, and the amount of a light absorbing component may be estimated from the estimated optical spectrum. FIG. 23 is a block diagram illustrating an example configuration of an image processing device according to the fifth embodiment.

As illustrated in FIG. 23, an image processing device 400 according to the fifth embodiment includes a computation unit 410 instead of the computation unit 140 illustrated in FIG. 4. The configurations and the operations of the respective components of the image processing device 400 other than the computation unit 410 are similar to those in the first embodiment.

The computation unit 410 includes a spectrum estimating unit 411 and an absorbance calculating unit 412 instead of the absorbance calculating unit 141 illustrated in FIG. 4.

The spectrum estimating unit 411 estimates the optical spectrum from an image based on image data read from the image data storage unit 132. More specifically, each of a plurality of pixels constituting an image is sequentially set to be a pixel to be estimated, and the estimated spectral transmittance T̂(x) at a point on an object corresponding to a point x on an image, which is the pixel to be estimated, is calculated from a matrix representation G(x) of the pixel value at the point x according to the following formula (29). The estimated spectral transmittance T̂(x) is a matrix having estimated transmittances t̂(x,λ) at respective wavelengths λ as elements. In addition, in the formula (29), a matrix W is an estimation operator used for Wiener estimation.

\hat{T}(x)=WG(x) (29)

The absorbance calculating unit 412 calculates absorbance at each wavelength λ from the estimated spectral transmittance T̂(x) calculated by the spectrum estimating unit 411. More specifically, the absorbance a(λ) at a wavelength λ is calculated by obtaining a logarithm of each of the estimated transmittances t̂(x,λ), which are elements of the estimated spectral transmittance T̂(x).

The operations of the component amount estimating unit 142 to the depth estimating unit 145 are similar to those in the first embodiment.

According to the fifth embodiment, estimation of a depth may also be performed on an image generated based on a broad signal value in the wavelength direction.

### Sixth Embodiment

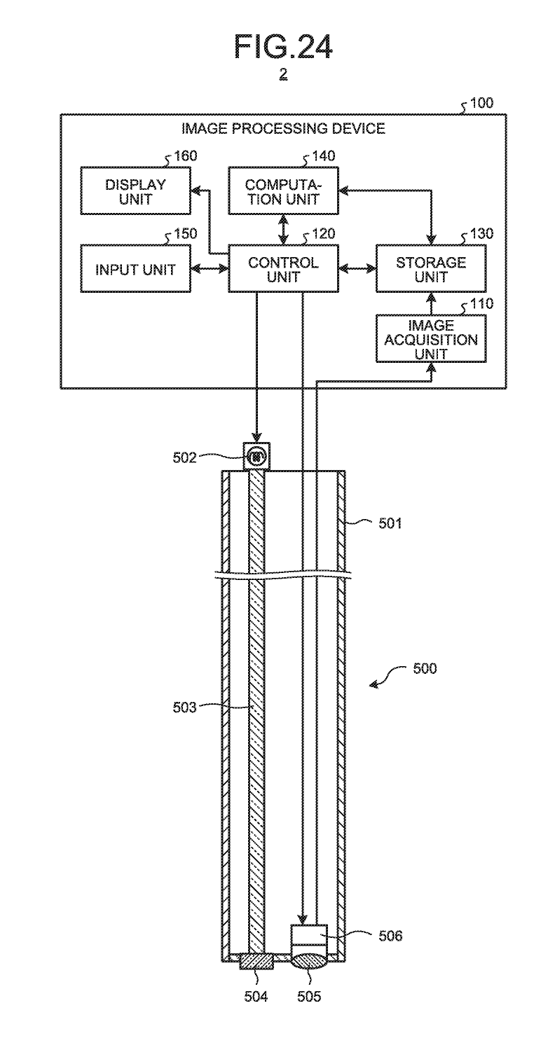

Next, a sixth embodiment will be described. FIG. 24 is a schematic diagram illustrating an example configuration of an imaging system according to the sixth embodiment. As illustrated in FIG. 24, an endoscope system 2 that is an imaging system according to the sixth embodiment includes an image processing device 100, and an endoscope apparatus 500 for generating an image of the inside of a lumen by inserting a distal end into a lumen of a living body and performing imaging.

The image processing device 100 performs predetermined image processing on an image generated by the endoscope apparatus 500, and generally controls the whole endoscope system 2. Note that the image processing devices described in the second to fifth embodiments may be used instead of the image processing device 100.

The endoscope apparatus 500 is a rigid endoscope in which an insertion part 501 to be inserted into a body cavity has rigidity, and includes the insertion part 501, and an illumination part 502 for generating illumination light to be emitted to the object from the distal end of the insertion part 501. The endoscope apparatus 500 and the image processing device 100 are connected with each other via a cable assembly of a plurality of signal lines through which electrical signals are transmitted and received.

The insertion part 501 is provided with a light guide 503 for guiding illumination light generated by the illumination part 502 to the distal end portion of the insertion part 501, an illumination optical system 504 for irradiating an object with the illumination light guided by the light guide 503, an objective lens 505 that is an imaging optical system for forming an image with light reflected by an object, and an imaging unit 506 for converting light with which an image is formed by the objective lens 505 into an electrical signal.

The illumination part 502 generates illumination light of each of wavelength bands into which a visible light range is divided under the control of the control unit 120. Illumination light generated by the illumination part 502 is emitted by the illumination optical system 504 via the light guide 503, and an object is irradiated with the emitted illumination light.

The imaging unit 506 performs imaging operation at a predetermined frame rate, generates image data by converting light with which an image is formed by the objective lens 505 into an electrical signal, and outputs the electrical signal to the image acquisition unit 110, under the control of the control unit 120.

Note that a light source for emitting white light may be provided instead of the illumination part 502, a plurality of optical filters having different spectral characteristics may be provided at the distal end portion of the insertion part 501, and multiband imaging may be performed by irradiating an object with white light and receiving light reflected by the object through an optical filter.

While an example in which an endoscope apparatus for a living body is applied as the imaging device from which the image processing devices according to the first to fifth embodiments acquire an image has been described in the sixth embodiment, an industrial endoscope apparatus may be applied. In addition, a flexible endoscope in which an insertion part to be inserted into a body cavity is bendable may be applied as the endoscope apparatus. Alternatively, a capsule endoscope to be introduced into a living body for performing imaging while moving inside the living body may be applied as the endoscope apparatus.

### Seventh Embodiment

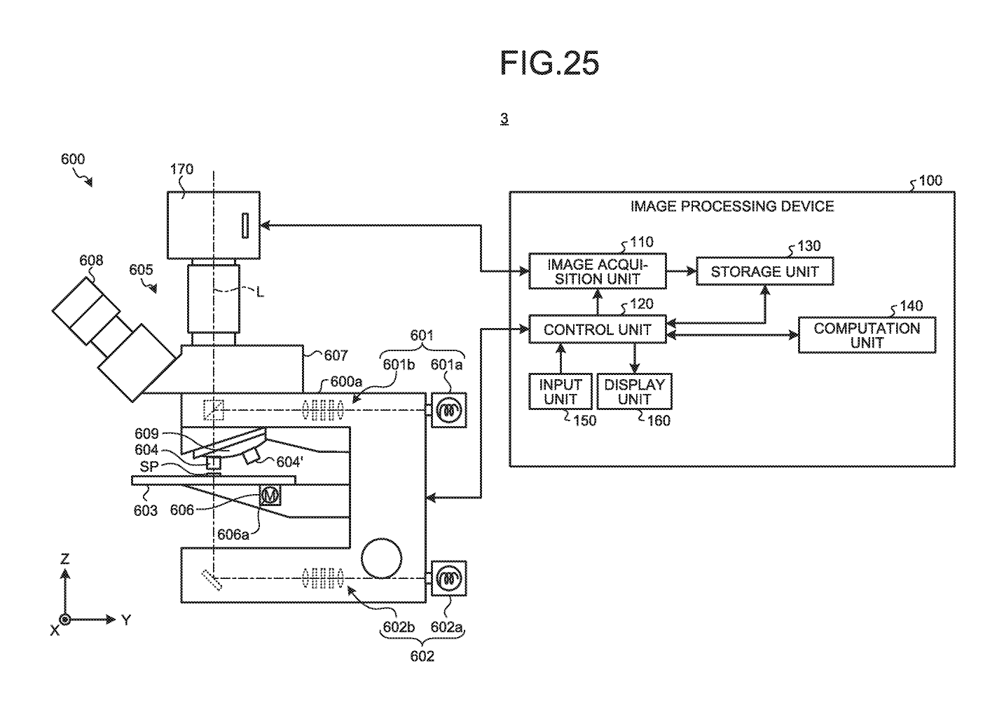

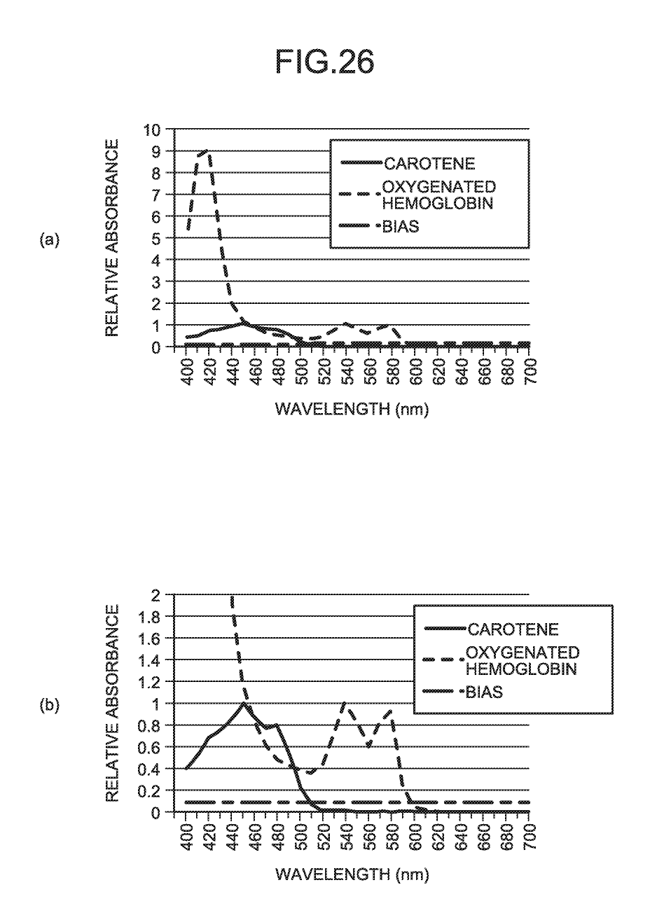

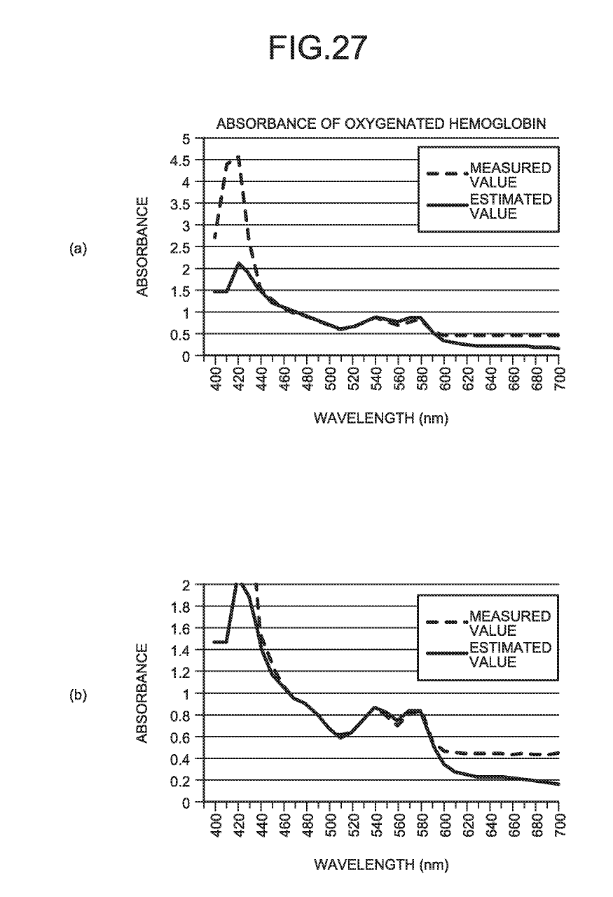

Next, a seventh embodiment will be described. FIG. 25 is a schematic diagram illustrating an example configuration of an imaging system according to the seventh embodiment. As illustrated in FIG. 25, a microscope system 3 that is an imaging system according to the seventh embodiment includes an image processing device 100, and a microscope apparatus 600 provided with an imaging device 170.

The imaging device 170 captures an object image enlarged by the microscope apparatus 600. The configuration of the imaging device 170 is not particularly limited, and an example of the configuration includes a monochromatic camera 171, a filter unit 172, and a tube lens 173 as illustrated in FIG. 5.

The image processing device 100 performs predetermined image processing on an image generated by the imaging device 170, and generally controls the whole microscope system 3. Note that the image processing devices described in the second to fifth embodiments may be used instead of the image processing device 100.

The microscope apparatus 600 has an arm 600a having substantially a C shape provided with an epi-illumination unit 601 and a transmitted-light illumination unit 602, a specimen stage 603 which is attached to the arm 600a and on which an object SP to be observed is placed, an objective lens 604 provided on one end side of a lens barrel 605 with a trinocular lens unit 607 therebetween to face the specimen stage 603, and a stage position changing unit 606 for moving the specimen stage 603.

The trinocular lens unit 607 separates light for observation of an object SP incident through the objective lens 604 to the imaging device 170 provided on the other end side of the lens barrel 605 and to an eyepiece unit 608, which will be described later. The eyepiece unit 608 is for a user to directly observe the object SP.

The epi-illumination unit 601 includes an epi-illumination light source 601a and an epi-illumination optical system 601b, and irradiates the object SP with epi-illumination light. The epi-illumination optical system 601b includes various optical members (a filter unit, a shutter, a field stop, an aperture diaphragm, etc.) for collecting illumination light emitted by the epi-illumination light source 601a and guiding the collected light toward an observation optical path L.

The transmitted-light illumination unit 602 includes a transmitted-light illumination light source 602a and a transmitted-light illumination optical system 602b, and irradiates the object SP with transmitted-light illumination light. The transmitted-light illumination optical system 602b includes various optical members (a filter unit, a shutter, a field stop, an aperture diaphragm, etc.) for collecting illumination light emitted by the transmitted-light illumination light source 602a and guiding the collected light toward the observation optical path L.

The objective lens 604 is attached to a revolver 609 capable of holding a plurality of objective lenses 604 and 604′, for example) having different magnification from each other. The imaging magnification may be changed in such a manner that the revolver 609 is rotated to switch between the objective lenses 604 and 604′ facing the specimen stage 603.

A zooming unit, including a plurality of zoom lenses and a drive unit for changing the positions of the zoom lenses, is provided inside the lens barrel 605 The zooming unit zooms in or out an object image within an imaging visual field by adjusting the positions of the zoom lenses.

The stage position changing unit 606 includes a drive unit 606a such as a stepping motor, and changes the imaging visual field by moving the position of the specimen stage 603 within an XY plane. In addition, the stage position changing unit 606 focuses the objective lens 604 on the object SP by moving the specimen stage 603 along a Z axis.

An enlarged image of the object SP generated by such a microscope apparatus 600 is subjected to multiband imaging by the imaging device 170, so that a color image of the object SP is displayed on the display unit 160.

The present disclosure is not limited to the first to seventh embodiments as described above, but the components disclosed in the first to seventh embodiments may be appropriately combined to achieve various inventions. For example, some of the components disclosed in the first to seventh embodiments may be excluded. Alternatively, components presented in different embodiments may be appropriately combined.

According to the present disclosure, the amounts of the light absorbing components are estimated and the estimation errors are calculated by using an absorbance in the first wavelength band in which a change in the light absorption characteristics of the light absorbing components contained in the specified tissue with a change in wavelength is small, and the depth is estimated by using an estimation error in the second wavelength band with shorter wavelengths than the first wavelength band, which reduces the influence of light absorbing components other than those contained in the specific tissue and allows estimation of the depth at which the specified tissue is present with high accuracy even when two or more kinds of tissue is present in an object.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An image processing device for estimating a depth of specified tissue included in an object based on an image obtained by capturing the object with light with a plurality of wavelengths, the image processing device comprising:
an absorbance calculating unit configured to calculate absorbances at the wavelengths based on pixel values of a plurality of pixels constituting the image;
a component amount estimating unit configured to estimate amounts of two or more kinds of light absorbing components contained respectively in two or more kinds of tissue including the specified tissue by using an absorbance in a first wavelength band among the absorbances at the wavelengths calculated by the absorbance calculating unit, the first wavelength band being a part of the wavelengths;
an estimation error calculating unit configured to calculate estimation errors caused by the component amount estimating unit; and
a depth estimating unit configured to estimate a depth at which the specified tissue is present in the object based on an estimation error in a second wavelength band with shorter wavelength than the first wavelength band among the estimation errors.

2. The image processing device according to claim 1, further comprising:
an estimation error normalizing unit configured to normalize the estimation errors calculated by the estimation error calculating unit by using an absorbance at a wavelength contained in the first wavelength band, wherein
the component amount estimating unit estimates the amounts of the two or more kinds of light absorbing components by using the absorbances calculated by the absorbance calculating unit, and
the depth estimating unit estimates the depth based on the estimation errors normalized by the estimation error normalizing unit.

3. The image processing device according to claim 1, further comprising:
an absorbance normalizing unit configured to normalize the absorbances calculated by the absorbance calculating unit by using an absorbance at a wavelength included in the first wavelength band to obtain normalized absorbances, wherein
the component amount estimating unit estimates two or more normalized amounts of components obtained by normalizing the amounts of two or more light absorbing components by using the normalized absorbances,
the estimation error calculating unit calculates normalized estimation errors by using the normalized absorbances and the two or more normalized amount of components, and
the depth estimating unit estimates the depth based on the normalized estimation errors.

4. The image processing device according to claim 1, wherein the first wavelength band is a wavelength band in which a change in light absorption characteristics of the light absorbing components contained in the specified tissue with a change in wavelength is small.

5. The image processing device according to claim 1, wherein the specified tissue is blood.

6. The image processing device according to claim 5, wherein
a light absorbing component contained in the specified tissue is oxygenated hemoglobin,
the first wavelength band ranges from 460 to 580 nm, and
the second wavelength band ranges from 400 to 440 nm.

7. The image processing device according to claim 2, wherein
the depth estimating unit estimates that the specified tissue is present at a surface of the object when the normalized estimation error in the second wavelength band is not larger than a threshold, and
the depth estimating unit estimates that the specified tissue is present at a depth from the surface of the object when the normalized estimation error in the second wavelength band is larger than the threshold.

8. The image processing device according to claim 1, further comprising:
a display unit configured to display the image; and
a control unit configured to determine a display mode for a region of the specified tissue in the image depending on a result of estimation by the depth estimating unit.

9. The image processing device according to claim 6, further comprising:
a second depth estimating unit configured to estimate a depth of tissue other than the specified tissue among the two or more kinds of tissue based on a result of estimation by the depth estimating unit.

10. The image processing device according to claim 9, wherein
the second depth estimating unit estimates that the tissue other than the specified tissue is present at a depth of the object when the depth estimating unit estimates that the specified tissue is present at a surface of the object, and
the second depth estimating unit estimates that the tissue other than the specified tissue is present at the surface of the object when the depth estimating unit estimates that the specified tissue is present at a depth of the object.

11. The image processing device according to claim 10, further comprising
a display unit configured to display the image; and
a display setting unit configured to set a display mode for a region of the tissue other than the specified tissue in the image depending on a result of estimation by the second depth estimating unit.

12. The image processing device according to claim 9, wherein the tissue other than the specified tissue is fat.

13. The image processing device according to claim 1, wherein the number of wavelengths is not smaller than the number of the light absorbing components.

14. The image processing device according to claim 1, further comprising:
a spectrum estimating unit configured to estimate an optical spectrum based on pixel values of a plurality of pixels constituting the image, wherein
the absorbance calculating unit calculates the absorbances at the wavelengths based on the optical spectrum estimated by the spectrum estimating unit.

15. An imaging system comprising:
the image processing device according to claim 1;
an illumination part configured to generate illumination light with which the object is irradiated;
an illumination optical system configured to emit the illumination light generated by the illumination part to the object;
an imaging optical system configured to form an image with light reflected by the object; and
an imaging unit configured to convert the light with which an image is formed by the imaging optical system into an electrical signal.

16. The imaging system according to claim 15, comprising:
an endoscope provided with the illumination optical system, the imaging optical system, and the imaging unit.

17. The imaging system according to claim 15, comprising:
a microscope apparatus provided with the illumination optical system, the imaging optical system, and the imaging unit.

18. An image processing method for estimating a depth of specified tissue included in an object based on an image obtained by capturing the object with light with the plurality of wavelengths, the image processing method comprising:
an absorbance calculating step of calculating absorbances at the wavelengths from pixel values of a plurality of pixels constituting the image;
a component amount estimating step of estimating amounts of two or more kinds of light absorbing components contained respectively in two or more kinds of tissue including the specified tissue by using an absorbance in a first wavelength band among the absorbances at the wavelengths calculated in the absorbance calculating step, the first wavelength band being a part of the wavelengths;
an estimation error calculating step of calculating estimation errors caused in the component amount estimating step; and
a depth estimating step of estimating a depth at which the specified tissue is present in the object based on an estimation error in a second wavelength band with shorter wavelength than the first wavelength band among the estimation errors.

19. A non-transitory computer-readable recording medium with an executable program stored thereon, the program being adapted to estimating a depth of specified tissue included in an object based on an image obtained by capturing the object with light with a plurality of wavelengths, and the program causing a processor to execute:
an absorbance calculating step of calculating absorbances at the wavelengths from pixel values of a plurality of pixels constituting the image;
a component amount estimating step of estimating amounts of two or more kinds of light absorbing components contained respectively in two or more kinds of tissue including the specified tissue by using an absorbance in a first wavelength band among the absorbances at the wavelengths calculated in the absorbance calculating step, the first wavelength band being a part of the wavelengths;
an estimation error calculating step of calculating estimation errors caused in the component amount estimating step; and
a depth estimating step of estimating a depth at which the specified tissue is present in the object based on an estimation error in a second wavelength band with shorter wavelength than the first wavelength band among the estimation errors.
